(19) United States
(12) Patent Application Publication
(10) Pub. No.: US 2024/0050500 A1
(43) Pub. Date: Feb. 15, 2024

(54) COMPOSITIONS AND METHODS FOR INHIBITING THE PROLIFERATION OF ENTEROTOXIGENIC BACTEROIDES FRAGILIS

(71) Applicant: iNtRON Biotechnology, Inc., Gyeonggi-do (KR)

(72) Inventors: Seong Jun YOON, Seoul (KR); Jee Soo SON, Seoul (KR); In Hwang KIM, Gyeonggi-do (KR); Hyoung Rok PAIK, Incheon (KR); Eun Kyoung OH, Gyeonggi-do (KR); Geun Woo LEE, Gyeonggi-do (KR); Soo Youn JUN, Seoul (KR); Sang Hyeon KANG, Seoul (KR)

(21) Appl. No.: 18/498,455

(22) Filed: Oct. 31, 2023

Related U.S. Application Data
(63) Continuation of application No. 17/227,287, filed on Apr. 10, 2021.

(51) Int. Cl. A61K 35/76; A61P 31/04

(52) U.S. Cl. CPC A61K 35/76; A61P 31/04

(57) ABSTRACT

A composition for preventing or treating an infection or disease caused by enterotoxigenic Bacteroides fragilis includes a Siphoviridae bacteriophage (Bac-FRP-5) having an ability to lyse the enterotoxigenic Bacteroides fragilis cells and a pharmaceutically acceptable carrier. A method for preventing or treating an infection or disease caused by enterotoxigenic Bacteroides fragilis includes administering to a subject a Siphoviridae bacteriophage and lysing the enterotoxigenic Bacteroides fragilis cells by the Siphoviridae bacteriophage.

### BACKGROUND OF THE INVENTION

### Field of the Invention

The present invention relates to compositions and methods for inhibiting the proliferation of enterotoxigenic Bacteroides fragilis, more specifically, a composition containing a Siphoviridae bacteriophage and a method of using the same.

### Discussion of the Related Art

Bacteroides species comprise nearly half of the fecal flora community and are host symbionts critical to host nutrition and mucosal and systemic immunity. Among Bacteroides species, Bacteroides fragilis (B. fragilis) strains are opportunistic pathogens. Enterotoxigenic B. fragilis (ETBF) can produce a proteolytic enterotoxin, named as B. fragilis enterotoxin (BFT), or fragilysin, that causes secretory diarrhea and colonic epithelial damage. ETBF emerged over the past 35 years as a global etiology of diarrheal disease in animals and humans that is accompanied by colitis (Clin. Microbiol. Rev. 22: 349-369, 2009). An association of ETBF with chronic intestinal disease has been established for more than 20 years and ETBF is also positively associated with ulcerative colitis and colonic neoplasia (Gut Pathog. 9: 53-59, 2017; BMC Canc. 19: 879-882, 2019).

In addition, ETBF may cause cancer such as colorectal cancer (CRC). CRC is one of the most common cancers, accounting for approximately 10% of all cancer cases and approximately 8% of all cancer deaths. BFT is known to bind to colonic epithelial cells (CECs) and to stimulate cleavage of the tumor suppressor protein, E-cadherin. E-cadherin cleavage increases intestinal barrier permeability and augments cell signaling via the β-catenin/Wnt pathway which is constitutively activated in essentially all CRC. As a result, BFT stimulates proliferation and migration of human colon cancer cells in vitro (Gastroenterology 124: 392-400, 2003). The ability of BFT to further activate the nuclear factor-kappaB (NF-κB) pathway inducing pro-inflammatory cytokine secretion by CECs and data indicating that specific pools of NF-κB foster the initiation and promotion of epithelial tumorigenesis led to the hypothesis that ETBF were pro-inflammatory, oncogenic colonic bacteria. This hypothesis was supported by a recent small study in Turkey suggesting that ETBF colonization is more frequent in CRC patients than in controls without CRC (Clin. Microbiol. Infect. 12: 782-786, 2006).

Generally, antibiotics are used for the treatment of infectious diseases of ETBF. Here, the effectiveness of antibiotics has been continuously decreasing due to the increase of antibiotic-resistant ETBF, and the development of effective methods other than currently prescribed antibiotics is required.

Recently, the use of bacteriophages as a countermeasure against bacterial infectious diseases has attracted considerable attention. Bacteriophages are very small microorganisms infecting bacteria, and are usually simply called “phages.” Once a bacteriophage infects a bacterial cell, the bacteriophage is proliferated inside the bacterial cell. After proliferation, the progeny of the bacteriophage destroys the bacterial cell wall and escapes from the host bacteria, suggesting that the bacteriophage has the ability to kill bacteria. The manner in which the bacteriophage infects bacteria is characterized by the very high specificity thereof, and thus the number of types of bacteriophages infecting a specific bacterium is limited. That is, a certain bacteriophage can infect only a specific bacterium, suggesting that a certain bacteriophage can kill only a specific bacterium and cannot harm other bacteria. Due to this bacteria specificity of bacteriophages, the bacteriophage confers antibacterial effects only upon target bacteria, but does not affect commensal bacteria in animals including human being. Conventional antibiotics, which have been widely used for bacterial treatment, incidentally influence many kinds of bacteria. This causes problems such as the disturbance of normal microflora. On the other hand, the use of bacteriophages does not disturb normal microflora, because the target bacterium is selectively killed. Hence, the bacteriophage may be utilized safely, which thus greatly lessens the probability of adverse actions in use compared to any other antibiotics.

Owing to the unique ability of bacteriophages to kill bacteria, bacteriophages have attracted attention as a potentially effective countermeasure against bacterial infections since their discovery, and there has been a lot of research related thereto.

Bacteriophages tend to be highly specific for bacteria. It has been shown that the attack of bacteriophage is specific, meaning that one species of bacteriophage targets only a single species of bacteria (or even a specific strain of one species). In addition, the antibacterial strength of bacteriophages may depend on the type of target bacterial strain. Therefore, it is necessary to collect many kinds of bacteriophages that are useful in order to get effective control of specific bacteria. Hence, in order to develop the effective bacteriophage utilization method in response to ETBF, many kinds of bacteriophages that exhibit antibacterial action against ETBF must be acquired. Furthermore, the resulting bacteriophages need to be screened as to whether or not they are superior to others from the aspect of antibacterial strength and spectrum.

### SUMMARY OF THE INVENTION

Accordingly, the present invention has been made keeping in mind the problems encountered in the related art and is intended to solve such problems.

In one embodiment, a composition for preventing or treating an infection or disease caused by ETBF includes: a Siphoviridae bacteriophage having an ability to lyse the ETBF cells, and a pharmaceutically acceptable carrier.

In another embodiment, the Siphoviridae bacteriophage has a genome including a sequence as set forth in SEQ ID NO: 1; or a genome that has (1) a sequence having at least 90% query cover with at least 90% identity to SEQ ID NO: 1, (2) a circular genome topology, and (3) 64 open reading frames.

In another embodiment, the Siphoviridae bacteriophage has a concentration of 1×10¹ pfu/ml to 1×10³⁰ pfu/ml or 1×10¹ pfu/g to 1×10³⁰ pfu/g.

In another embodiment, the Siphoviridae bacteriophage has a concentration of 1×10⁴ pfu/ml to 1×10¹⁵ pfu/ml or 1×10⁴ pfu/g to 1×10¹⁵ pfu/g.

In another embodiment, the pharmaceutically acceptable carrier is lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, or mineral oil.

In another embodiment, the composition further includes one or more selected from the group consisting of a lubricant, a wetting agent, a sweetener, a flavor, an emulsifier, a suspending agent, and a preservative.

In another embodiment, the infection or disease caused by ETBF is acute and chronic intestinal disease, bacteremia, diarrhea, colitis, colonic neoplasia, or cancer. The cancer is colorectal cancer or colon cancer, but is not limited thereto.

In another embodiment, the composition is a solution, suspension, emulsion in oil, water-soluble medium, extract, powder, granule, tablet, or capsule.

In another embodiment, the composition further includes a second bacteriophage having an ability to lyse ETBF bacterial species and the second bacteriophage has a genome that has a sequence having less than 90% query cover with at least 90% identity to SEQ ID NO: 1.

In another embodiment, the Siphoviridae bacteriophage has major structural proteins in the sizes of approximately 42 kDa, 48 kDa, 63 kDa, 65 kDa, 75 kDa, 88 kDa, and 118 kDa.

In another embodiment, the Siphoviridae bacteriophage has a latent period of 10-100 minutes and a burst size of 150-540 PFU/infected cell.

In another embodiment, the latent period is 40-80 minutes and the burst size of 200-450 PFU/infected cell.

In one embodiment, a method for preventing or treating an infection or disease caused by ETBF includes administering to a subject a Siphoviridae bacteriophage; and lysing the ETBF by the Siphoviridae bacteriophage.

In another embodiment, the Siphoviridae bacteriophage includes a sequence as set forth in SEQ ID NO: 1.

In another embodiment, the Siphoviridae bacteriophage has a concentration of 1×10¹ pfu/ml to 1×10³ pfu/ml or 1×10¹ pfu/g to 1×10³ pfu/g.

In another embodiment, the Siphoviridae bacteriophage has a concentration of 1×10⁴ pfu/ml to 1×10¹⁵ pfu/ml or 1×10⁴ pfu/g to 1×10¹⁵ pfu/g.

It is to be understood that both the foregoing general description and the following detailed description are exemplary and explanatory and are intended to provide further explanation of the invention as claimed.

### ADVANTAGEOUS EFFECTS OF INVENTION

The compositions and methods for inhibiting the proliferation of ETBF, of the present application have high specificity against ETBF, compared with conventional compositions and methods based on antibiotics. The compositions can be used for preventing or treating ETBF infections without affecting other useful commensal bacteria and have fewer side effects. In general, when antibiotics are used, commensal bacteria are also damaged, thus entailing various side effects owing to the use thereof. Meanwhile, each antibacterial property of the bacteriophages such as antibacterial strength and spectrum (host range) are different in the case of bacteriophages exhibiting antibacterial activity against the same bacterial species and bacteriophages are usually effective only on some bacterial strains within the same bacterial species. Thus, the compositions and methods of the present application provide different effects in its industrial applications.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 is an electron micrograph showing the morphology of the bacteriophage Bac-FRP-5.

FIG. 2 is a result of the analysis for major structural proteins of bacteriophage Bac-FRP-5.

FIG. 3 is a photograph showing the results of an experiment on the ability of the bacteriophage Bac-FRP-5 to kill ETBF. The clear zone is a plaque formed by lysis of the target bacteria.

FIG. 4 is the one-step growth curve of bacteriophage Bac-FRP-5.

### DETAILED DESCRIPTION OF THE ILLUSTRATED EMBODIMENTS

Reference will now be made in detail to embodiments of the present invention, example of which is illustrated in the accompanying drawings.

In accordance with one aspect of the present invention, the present invention provides a Siphoviridae bacteriophage, named as Bac-FRP-5, which has the ability to specifically kill ETBF and has a genome including a sequence as set forth in SEQ ID NO: 1. In some embodiment, the Siphoviridae bacteriophage contains a genome that has all the following characteristics: 1) including a sequence having at least 90% query cover with at least 90% identity to SEQ ID NO: 1, 2) having a circular genome topology, and 3) having 64 open reading frames; a genome that has all the following characteristics: 1) including a sequence having at least 92% query cover with at least 90% identity to SEQ ID NO: 1, 2) having the circular genome topology, and 3) having 64 open reading frames; a genome that has all the following characteristics: 1) including a sequence having at least 94% query cover with at least 90% identity to SEQ ID NO: 1, 2) having the circular genome topology, and 3) having 64 open reading frames; or a genome that has all the following characteristics: 1) including a sequence having at least 96% query cover with at least 90% identity to SEQ ID NO: 1, 2) having the circular genome topology, and 3) having 64 open reading frames.

The present invention also provides a method for preventing and treating infections or diseases caused by ETBF using a composition including the same as an active ingredient.

The bacteriophage Bac-FRP-5 was isolated by the present inventors and then deposited at Korea Collection for Type Cultures, Korea Research Institute of Bioscience and Biotechnology on Dec. 9, 2020 (Accession number: KCTC 14403BP).

The molecular weight of major structural proteins of the bacteriophage Bac-FRP-5 is approximately 42 kDa, 48 kDa, 63 kDa, 65 kDa, 75 kDa, 88 kDa, and 118 kDa.

The latent period and burst size of the bacteriophage Bac-FRP-5 are 10-100 minutes and 150-540 PFU/infected cell, respectively, preferably 40-80 minutes and 200-450 PFU/infected cell, respectively, but are not limited thereto.

Also, the present invention provides a composition applicable for the prevention or treatment of infections or diseases caused by ETBF, which include the bacteriophage Bac-FRP-5 as an active ingredient.

Because the bacteriophage Bac-FRP-5 included in the composition of the present invention kills ETBF effectively, it is considered effective in the prevention of ETBF infections or treatment of diseases caused by ETBF. Therefore, the composition of the present invention is capable of being utilized for the prevention and treatment of diseases caused by ETBF.

The diseases caused by ETBF in the present invention include acute and chronic intestinal disease, bacteremia, diarrhea, colitis, colonic neoplasia, or cancer, but are not limited thereto.

The pharmaceutically acceptable carrier included in the composition of the present invention is one that is generally used for the preparation of a pharmaceutical formulation, and examples thereof include lactose, dextrose, sucrose, sorbitol, mannitol, starch, acacia rubber, calcium phosphate, alginate, gelatin, calcium silicate, microcrystalline cellulose, polyvinyl pyrrolidone, cellulose, water, syrup, methylcellulose, methylhydroxybenzoate, propylhydroxybenzoate, talc, magnesium stearate, and mineral oil, but are not limited thereto. The composition of the present invention may additionally include lubricants, wetting agents, sweeteners, flavors, emulsifiers, suspending agents, and preservatives, in addition to the above ingredients.

In the composition of the present invention, the bacteriophage Bac-FRP-5 is included as an active ingredient. The bacteriophage Bac-FRP-5 is included at a concentration of 1×10¹ pfu/ml to 1×10³ pfu/ml or 1×10¹ pfu/g to 1×10³⁰ pfu/g, and preferably at a concentration of 1×10⁴ pfu/ml to 1×10¹⁵ pfu/ml or 1×10⁴ pfu/g to 1×10¹⁵ pfu/g.

The composition of the present invention can be formulated according to a method that can be easily performed by those of ordinary skill in the art to which the present invention pertains using a pharmaceutically acceptable carrier and/or excipient in the form of a unit dose or in a multi-dose container. Then, the formulation may be in the form of a solution, suspension, or emulsion in oil or a water-soluble medium, extract, powder, granule, tablet, or capsule. A dispersing agent or stabilizer may be additionally included.

In order to improve the effectiveness of above purpose, bacteriophages that have antibacterial activity against non-ETBF bacterial species may be further included in the composition of the present invention. In addition, other kinds of bacteriophages that have antibacterial activity against ETBF may be further included in the composition of the present invention. These bacteriophages may be additionally included so as to maximize antibacterial effects, because each antibacterial property of the bacteriophages such as antibacterial strength and spectrum (host range) are different in the case of bacteriophages exhibiting antibacterial activity against the same bacterial species.

In this description, the terms “prevention” and “prevent” indicate (i) to block ETBF infections; and (ii) to inhibit the progression of diseases caused by ETBF infections.

In this description, the terms “treatment” and “treat” indicate all actions that (i) suppress diseases caused by ETBF; and (ii) alleviate the pathological condition of the diseases caused by ETBF.

In this description, the terms “diseases caused by ETBF” and “ETBF infections” indicate acute and chronic intestinal disease, bacteremia, diarrhea, colitis, colonic neoplasia, or cancer, but are not limited thereto.

In this description, the term “Latent period” indicates the time taken by a bacteriophage particle to reproduce inside an infected host cell.

In this description, the term “Burst size” indicates the number of bacteriophages produced per infected bacterium.

In this description, the terms “isolate,” “isolating,” and “isolated” indicate actions which isolate bacteriophages from nature by applying diverse experimental techniques and which secure characteristics that can distinguish the target bacteriophage from others, and further include the action of proliferating the target bacteriophage using bioengineering techniques so that the target bacteriophage is industrially applicable.

In this description, the terms “query cover” and “identity” are related to BLAST (Basic Local Alignment Search Tool) which is an online search tool provided by NCBI (National Center for Biotechnology Information).

In this description, the query cover is a number that describes how much of the query sequence (i.e., the sequence of genome of bacteriophage Bac-FRP-5) is covered by the target sequence (i.e., the sequence of genome of the previously reported bacteriophage). If the target sequence in the database spans the whole query sequence, then the query cover is 100%. This tells us how long the sequences are, relative to each other.

In this description, the term “identity” or “sequence identity” was measured for “query cover,” and is a number that describes how similar the query sequence (i.e., the sequence of genome of bacteriophage Bac-FRP-5) is to the target sequence (i.e., the sequence of genome of the previously reported bacteriophage). More specifically, the terms “identity” or “sequence identity” refers to the percentage of identical nucleotides in the spanned sequence part of the target sequence (i.e., the sequence of genome of the previously reported bacteriophage) or the query sequence (i.e., the sequence of genome of bacteriophage Bac-FRP-5) when the query sequence (i.e., the sequence of genome of bacteriophage Bac-FRP-5) and the target sequence (i.e., the sequence of genome of the previously reported bacteriophage) are analyzed by BLAST alignment analysis. The higher the percent identity is, the more significant the match is. From above definitions for “query cover” and “sequence identity,” it will be obvious for the skilled one in the art that the differences of “query cover” and/or “sequence identity” between genomes of two similar bacteriophages make the differences of ORF (open reading frame)'s numbers arranged in the two genomes, then results in the discriminative characteristics (including the range of target strain and strength of antibacterial activity) of two similar bacteriophages.

In this description, the term “Second Bacteriophage” is any bacteriophage that has the ability to specifically kill ETBF and has a genome that has a sequence having less than 90% query cover with at least 90% identity to SEQ ID NO: 1 and has different characteristics from bacteriophage Bac-FRP-5 in terms of the genome topology and the number of ORFs, wherein the genome topology of the Second Bacteriophage is linear form.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1: ISOLATION OF BACTERIOPHAGE CAPABLE OF KILLING ETBF

Samples were collected from environmental or clinical samples to isolate the bacteriophage capable of killing ETBF. Here, the ETBF strains used for the bacteriophage isolation had been previously isolated and identified as ETBF by the present inventors.

The procedure for isolating the bacteriophage is described in detail hereinafter. The collected sample was added to a BHIB (Brain Heart Infusion Broth) culture medium (calf brain infusion from 200 g, 7.7 g/L; beef heart infusion from 250 g, 9.8 g/L; proteose peptone, 10 g/L; dextrose, 2 g/L; sodium chloride, 5 g/L; disodium phosphate, 2.5 g/L) inoculated with ETBF at a ratio of 1/100, followed by stationary culture at 37 C for two days under anaerobic condition. Upon completion of the culture, centrifugation was performed at 8,000 rpm for 20 minutes and a supernatant was recovered. The recovered supernatant was inoculated with ETBF at a ratio of 1/100, followed by stationary culture at 37 C for two days under anaerobic condition. When the sample contained the bacteriophage, the above procedure was repeated a total of 5 times in order to sufficiently increase the number (titer) of the bacteriophage. After repeating the procedure 5 times, the culture solution was subjected to centrifugation at 8,000 rpm for 20 minutes. After the centrifugation, the recovered supernatant was filtered using a 0.45 m filter. The obtained filtrate was used in a typical spot assay for examining whether or not a bacteriophage capable of killing ETBF was included therein.

The spot assay was performed as follows: BHIB culture medium was inoculated with ETBF at a ratio of 1/100, followed by stationary culture at 37° C. for two days under anaerobic condition. 2 ml (OD₆₀₀ of 1.5) of the culture solution of ETBF prepared above was spread on BHIA (calf brain infusion from 200 g, 7.7 g/L; beef heart infusion from 250 g, 9.8 g/L; proteose peptone, 10 g/L; dextrose, 2 g/L; sodium chloride, 5 g/L; disodium phosphate, 2.5 g/L; agar, 15 g/L) plate. The plate was left on a clean bench for about 30 minutes to dry the spread solution. After drying, 10 l of the prepared filtrate was spotted onto the plate culture medium on which ETBF was spread and then left to dry for about 30 minutes. After drying, the plate culture medium that was subjected to spotting was incubated at 37 C for two days under anaerobic condition, and then examined for the formation of clear zones at the positions where the filtrate was dropped. In the case of the filtrate generated a clear zone, it is judged that the bacteriophage capable of killing ETBF is included therein. Through the above examination, the filtrate containing the bacteriophage having the ability to kill ETBF could be obtained.

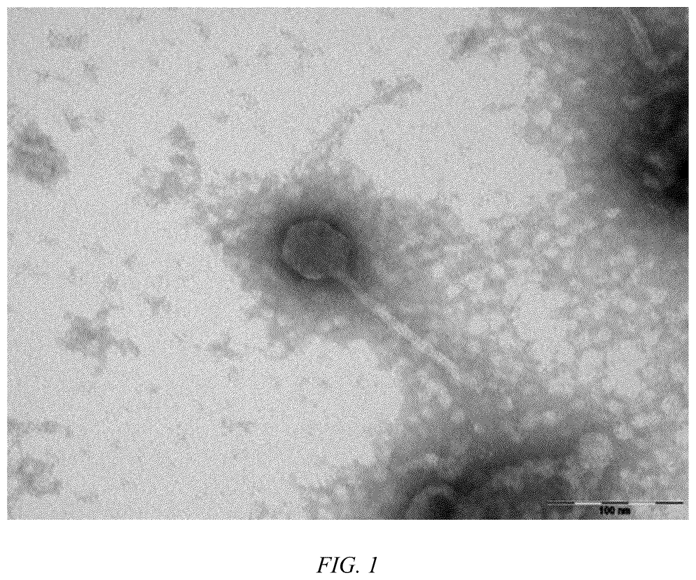

The pure bacteriophage was isolated from the filtrate confirmed above to have the bacteriophage capable of killing ETBF. A conventional plaque assay was used to isolate the pure bacteriophage. In detail, a plaque formed in the course of the plaque assay was recovered using a sterilized tip, which was then added to the culture solution of ETBF, followed by culturing at 37 C two days under anaerobic condition. After the culturing, centrifugation was performed at 8,000 rpm for 20 minutes to obtain a supernatant. The ETBF culture solution was added to the obtained supernatant at a volume ratio of 1/50, followed by culturing at 37 C for 2 days under anaerobic condition. In order to increase the number of bacteriophages, the above procedure was repeated at least 5 times. Then, centrifugation was performed at 8,000 rpm for 20 minutes in order to obtain the final supernatant. A plaque assay was further performed using the resulting supernatant. In general, the isolation of a pure bacteriophage is not completed through a single iteration of a procedure, so the above procedure was repeated using the resulting plaque formed above. After at least 5 repetitions of the procedure, a solution containing the pure bacteriophage was obtained. The procedure for isolating the pure bacteriophage was generally repeated until the generated plaques became similar to each other in size and morphology. In addition, final isolation of the pure bacteriophage was confirmed using electron microscopy. The above procedure was repeated until the isolation of the pure bacteriophage was confirmed using electron microscopy. The electron microscopy was performed according to a conventional method. Briefly, the solution containing the pure bacteriophage was loaded on a copper grid, followed by negative staining with 2% uranyl acetate and drying. The morphology thereof was then observed using a transmission electron microscope. The electron micrograph of the pure bacteriophage that was isolated is shown in FIG. 1. Based on the morphological characteristics, the novel bacteriophage isolated above was confirmed to belong to the Siphoviridae bacteriophage.

The solution containing the pure bacteriophage confirmed above was subjected to the following purification process. The ETBF culture solution was added to the solution containing the pure bacteriophage at a volume ratio of 1/50 based on the total volume of the bacteriophage solution, followed by further culturing for two days under anaerobic condition. After the culturing, centrifugation was performed at 8,000 rpm for 20 minutes to obtain a supernatant. This procedure was repeated a total of 5 times in order to obtain a solution containing sufficient numbers of the bacteriophage. The supernatant obtained from the final centrifugation was filtered using a 0.45 m filter, followed by a conventional polyethylene glycol (PEG) precipitation process. Specifically, PEG and NaCl were added to 100 ml of the filtrate until reaching 10% PEG 8000/0.5 M NaCl, and then left at 4 C for 2 to 3 hours. Thereafter, centrifugation was performed at 8,000 rpm for 30 minutes to obtain the bacteriophage precipitate. The resulting bacteriophage precipitate was suspended in 5 ml of a buffer (10 mM Tris-HCl, 10 mM MgSO₄, 0.1% gelatin, pH 8.0). The resulting material was referred to as a bacteriophage suspension or bacteriophage solution.

As a result, the pure bacteriophage purified above was collected, was named the bacteriophage Bac-FRP-5, and then deposited at Korea Collection for Type Culture, Korea Research Institute of Bioscience and Biotechnology on Dec. 9, 2020 (Accession number: KCTC 14403BP).

### EXAMPLE 2: SEPARATION AND SEQUENCE ANALYSIS OF GENOME OF BACTERIOPHAGE Bac-FRP-5

The genome of the bacteriophage Bac-FRP-5 was separated as follows. The genome was separated from the bacteriophage suspension obtained using the same method as in Example 1. First, in order to remove DNA and RNA of ETBF included in the suspension, 200 U of each of DNase I and RNase A was added to 10 ml of the bacteriophage suspension and then left at 37 C for 30 minutes. After being left for 30 minutes, in order to stop the DNase I and RNase A activity, 500 l of 0.5 M ethylenediaminetetraacetic acid (EDTA) was added thereto and then left for 10 minutes. In addition, the resulting mixture was further left at 65 C for 10 minutes, and 100 l of proteinase K (20 mg/ml) was then added thereto so as to break the outer wall of the bacteriophage, followed by reaction at 37 C for 20 minutes. After that, 500 l of 10% sodium dodecyl sulfate (SDS) was added thereto, followed by reaction at 65 C for 1 hour. After reaction for 1 hour, 10 ml of the solution of phenol:chloroform:isoamyl alcohol, mixed at a component ratio of 25:24:1, was added to the reaction solution, followed by mixing thoroughly. In addition, the resulting mixture was subjected to centrifugation at 13,000 rpm for 15 minutes to separate layers. Among the separated layers, the upper layer was selected, and isopropyl alcohol was added thereto at a volume ratio of 1.5, followed by centrifugation at 13,000 rpm for 10 minutes in order to precipitate the genome. After collecting the precipitate, 70% ethanol was added to the precipitate, followed by centrifugation at 13,000 rpm for 10 minutes to wash the precipitate. The washed precipitate was recovered, vacuum-dried and then dissolved in 100 1 of water. This procedure was repeated to obtain a sufficient amount of the genome of the bacteriophage Bac-FRP-5.

Information on the sequence of the genome of the bacteriophage Bac-FRP-5 obtained above was secured by performing next-generation sequencing analysis using Illumina Mi-Seq equipment from the National Instrumentation Center for Environmental Management, Seoul National University. The finally analyzed genome of the bacteriophage Bac-FRP-5 had a size of 46,506 bp, and the sequence of whole genome was expressed by SEQ ID NO: 1.

The homology (similarity) of the bacteriophage Bac-FRP-5 genomic sequence obtained above with previously reported bacteriophage genomic sequences was investigated using BLAST investigation, the genomic sequence of the bacteriophage Bac-FRP-5 was found to have a relatively high homology with the sequence of the Bacteroides bacteriophage B40-8 culture-collection ATCC:51477-B1 (Genbank Accession No. FJ008913.1) (query cover: 34%, sequence identity: 87.71%). In addition, the number of open reading frames (ORFs) on the bacteriophage Bac-FRP-5 genome is 64, whereas Bacteroides bacteriophage B40-8 culture-collection ATCC:51477-B1 has 46 open reading frames.

Based upon this result, it is concluded that the bacteriophage Bac-FRP-5 must be a novel bacteriophage different from conventionally reported bacteriophages. Further, since the antibacterial strength and spectrum of bacteriophages typically depend on the type of bacteriophage, it is considered that the bacteriophage Bac-FRP-5 can provide antibacterial activity different from that of any other bacteriophages reported previously.

### EXAMPLE 3: ANALYSIS OF THE MAJOR STRUCTURAL PROTEINS OF BACTERIOPHAGE Bac-FRP-5

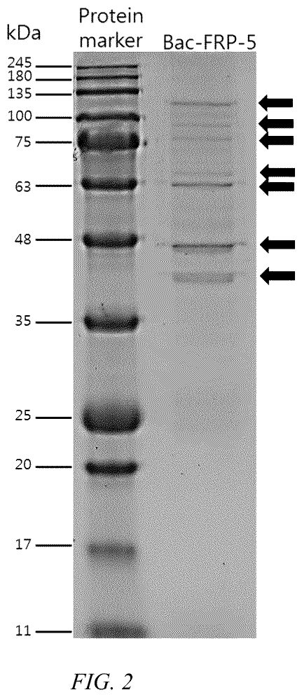

One-dimensional electrophoresis was performed to analyze the major structural proteins of the bacteriophage Bac-FRP-5. To obtain the proteins constituting the outer wall of the bacteriophage Bac-FRP-5, 200 μl of the bacteriophage suspension prepared in Example 1 was mixed with 800 μl of acetone, which was vortexed vigorously. The mixture stood at −20 C for 10 minutes. Centrifugation was performed at 13,000 rpm at 4 C for 20 minutes to eliminate supernatant, followed by air drying. The precipitate was resuspended in 50 μl of electrophoresis sample buffer (5×), which was then boiled for 5 minutes. The prepared sample was analyzed by one-dimensional electrophoresis. As a result, as shown in FIG. 2, the major structural proteins in the sizes of approximately 42 kDa, 48 kDa, 63 kDa, 65 kDa, 75 kDa, 88 kDa, and 118 kDa were confirmed.

### EXAMPLE 4: INVESTIGATION OF ABILITY OF BACTERIOPHAGE Bac-FRP-5 TO KILL ETBF

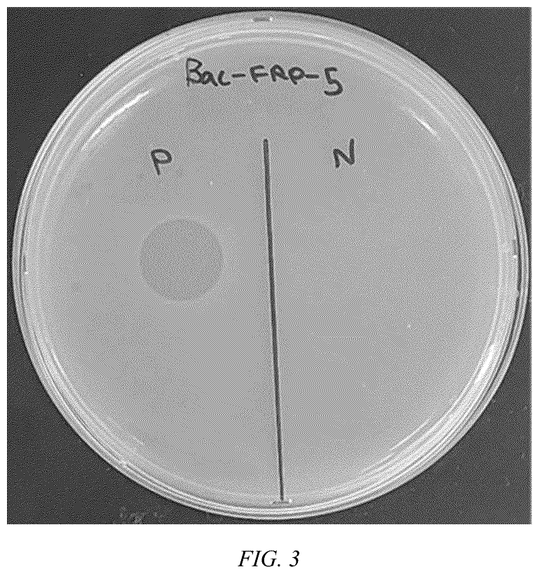

The ability of bacteriophage Bac-FRP-5 to kill ETBF was investigated. In order to investigate the killing ability, the formation of clear zones was observed using the spot assay in the same manner as described in Example 1. A total of 5 strains that had been identified as ETBF strains were used as ETBF for the investigation of killing ability. The bacteriophage Bac-FRP-5 had the ability to lyse and kill a total of 4 strains among 5 strains of ETBF as the experimental target. The experimental result thereof is presented in Table 1 and the representative result is shown in FIG. 3.

TABLE 1
Test of antibacterial activity of bacteriophage Bac-FRP-5

| Tested ETBF strain | Test result |
|---|---|
| Bacteroides fragilis CCARM 18105 | + |
| Bacteroides fragilis CCARM 18106 | + |
| Bacteroides fragilis CCARM 18107 | − |
| Bacteroides fragilis CCARM 18110 | + |
| Bacteroides fragilis CCARM 18111 | + |

* +: clear lytic activity, −: no lytic activity;
CCARM: Culture Collection of Antimicrobial Resistant Microbes (Seoul, Korea)

Meanwhile, the ability of the bacteriophage Bac-FRP-5 to kill Bordetella bronchiseptica, Enterococcus faecalis, Enterococcus faecium, Staphylococcus aureus, Streptococcus pneumonia, E. coli and Pseudomonas aeruginosa was also investigated in a separate experiment. As a result, the bacteriophage Bac-FRP-5 did not have the ability to kill these bacteria.

Therefore, it is confirmed that the bacteriophage Bac-FRP-5 has strong ability to kill ETBF and a broad antibacterial spectrum against ETBF, suggesting that the bacteriophage Bac-FRP-5 can be used as an active ingredient of the composition for preventing and treating ETBF infections.

### EXAMPLE 5: GROWTH CHARACTERISTIC OF BACTERIOPHAGE Bac-FRP-5

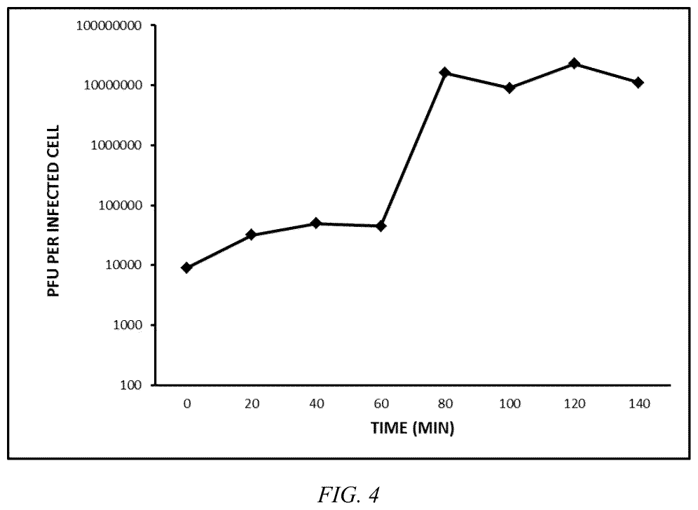

The growth characteristics of bacteriophage Bac-FRP-5 was analyzed by one-step growth curve analysis. One-step growth curve analysis of bacteriophage Bac-FRP-5 was performed as follows: 50 ml of BHIB (Brain heart infusion broth, Difco) culture medium was inoculated with ETBF at a ratio of 1/100 and followed by stationary culture until exponential phase (OD₆₀₀=0.4-0.5) under anaerobic condition. Upon completion of the culture, centrifugation was performed at 8,000 rpm for 5 min and a bacterial cell pellet was recovered. The recovered pellet was suspended in 50 ml of BHIB. The resulting material may be referred to as a bacterial suspension. The bacteriophage Bac-FRP-5 was mixed with the bacterial suspension at a multiplicity of infection (MOI) of 0.1 and incubated at room temperature for 10 min, and then centrifuged at 12,000 rpm for 30 seconds. After supernatants were removed, the pellets containing bacteriophage-infected bacterial cells were suspended in 50 ml of BHIB and incubated at 37 C without shaking. Aliquots were taken at 20 min intervals for 140 min, and the titers in the aliquots were immediately determined by the conventional plaque assay (FIG. 4).

The latent period of bacteriophage Bac-FRP-5 was estimated to be approximately 60±10 min with average burst size of about 320±100 pfu/infected cell.

### EXAMPLE 6: EXPERIMENTAL EXAMPLE REGARDING PREVENTION OF ETBF INFECTION USING BACTERIOPHAGE Bac-FRP-5

100 l of a bacteriophage Bac-FRP-5 suspension (1×10⁸ pfu/ml) was added to a tube containing 9 ml of a BHIB culture medium. To another tube containing 9 ml of a BHIB culture medium, only the same amount of BHIB culture medium was further added. A culture solution of ETBF strain (CCARM 18106) was then added to each tube so that absorbance reached about 0.5 at 600 nm. After ETBF was added, the tubes were transferred to an incubator at 37 C, followed by stationary culture, during which the growth of ETBF was observed. As presented in Table 2, it was observed that the growth of ETBF was inhibited in the tube to which the bacteriophage Bac-FRP-5 suspension was added, while the growth of ETBF was not inhibited in the tube to which the bacteriophage suspension was not added.

TABLE 2
Test for bacterial growth inhibition of bacteriophage Bac-FRP-5

| | OD₆₀₀ | | |
|---|---|---|---|
| | 0 minutes after | 120 minutes after | 240 minutes after |
| | initiation of | initiation of | initiation of |
| Classification | cultivation | cultivation | cultivation |
| Bacteriophage | 0.5 | 0.6 | 0.7 |
| suspension was | | | |
| not added | | | |
| Bacteriophage | 0.5 | 0.4 | 0.3 |
| suspension was | | | |
| added | | | |

The above results indicate that the bacteriophage Bac-FRP-5 of the present invention not only inhibits the growth of ETBF but also has the ability to kill ETBF. Therefore, it is concluded that the bacteriophage Bac-FRP-5 can be used as an active ingredient of the composition for preventing the ETBF infections.

### EXAMPLE 7: PREVENTIVE EFFECT OF BACTERIOPHAGE Bac-FRP-5 ON THE INFECTIONS OF ETBF IN ANIMAL MODEL

Preventive effect of the bacteriophage Bac-FRP-5 on weaning pigs affected by ETBF was investigated. 4 weaning pigs at 25 days of age were grouped together; total 2 groups of pigs were raised in each pig pen (1.1 m×1.0 m). Heating system was furnished and the surrounding environment was controlled. The temperature and the humidity of the pig pen were controlled consistently and the floor was cleaned every day. From the Pt day of the experiment, pigs of the experimental group (adding the bacteriophage) were fed with feeds adding the bacteriophage Bac-FRP-5 at 1×10⁸ pfu/g according to the conventional feed supply procedure, while pigs of the control group (without adding the bacteriophage) were fed with the same feed without adding the bacteriophage Bac-FRP-5 according to the conventional procedure. From the 7^{th} day of the experiment, the feeds of both groups were contaminated with 1×10⁸ cfu/g of ETBF for 2 days and thereafter provided twice a day respectively for the experimental and the control groups so as to bring about the infections of ETBF. The administered ETBF suspension was prepared as follows: ETBF strain (CCARM 18110) was anaerobically cultured at 37 C for two days using a BHIB culture medium, after which the bacteria were isolated and adjusted to 10⁹ CFU/ml using physiological saline (pH 7.2). From the next day after providing contaminated feeds for 2 days (the 9^{th} day of the experiment), pigs of the experimental group (adding the bacteriophage) were fed again with the feeds adding the bacteriophage Bac-FRP-5 at 1×10⁸ pfu/g without contaminating ETBF according to the conventional feed supply procedure as before, while pigs of the control group (without adding the bacteriophage) were fed with the same feed without adding the bacteriophage according to the conventional procedure. From the 9^{th} day of the experiment, diarrhea was examined in all test animals on a daily basis. The extent of diarrhea was determined by measuring according to a diarrhea index. The diarrhea index was measured using a commonly used Fecal Consistency (FC) score (normal: 0, soft stool: 1, loose diarrhea: 2, severe diarrhea: 3). The results are shown in Table 3.

TABLE 3
Fecal Consistency score

| | Fecal Consistency score | | | | | |
|---|---|---|---|---|---|---|
| | D9 | D10 | D11 | D12 | D13 | D14 |
| Control group | 2.25 | 2.0 | 1.75 | 1.5 | 1.25 | 1.0 |
| (bacteriophage | | | | | | |
| suspension was | | | | | | |
| not administered) | | | | | | |
| Experimental group | 1.0 | 0.75 | 0.25 | 0.25 | 0 | 0 |
| (bacteriophage | | | | | | |
| suspension was | | | | | | |
| administered) | | | | | | |

From the above results, it is confirmed that the bacteriophage Bac-FRP-5 of the present invention could be very effective to suppress the infections of ETBF.

### EXAMPLE 8: EXAMPLE OF TREATMENT OF INFECTIOUS DISEASES OF ETBF USING BACTERIOPHAGE Bac-FRP-5

The therapeutic effect of the bacteriophage Bac-FRP-5 on diseases caused by ETBF was evaluated as follows: 40 of 8-week-old mice were divided into a total of 2 groups of 20 mice per group, after which subgroups of 5 mice each were separately reared in individual experimental mouse cages, and the experiment was performed for 7 days. On the second day of the experiment, 0.1 ml of an ETBF suspension was administered to all mice through intraperitoneal injection. The administered ETBF suspension was prepared as follows: ETBF strain (CCARM 18110) was anaerobically cultured at 37 C for two days using a BHIB culture medium, after which the bacteria were isolated and adjusted to 10⁹ CFU/ml using physiological saline (pH 7.2). At 2 hr after administration of ETBF, 10⁹ pfu of bacteriophage Bac-FRP-5 was administered through intraperitoneal injection to mice in the experimental group (administered with the bacteriophage suspension). 0.1 ml of saline was administered through intraperitoneal injection to mice in the control group (not administered with the bacteriophage suspension). Both the control and experimental groups were equally fed with feed and drinking water. Whether or not the mice survived was observed daily starting from the administration of ETBF until the end of the test. The results are shown in Table 4 below.

TABLE 4
Survival rate

| | Survival rate (%) | | | | | |
|---|---|---|---|---|---|---|
| | D2 | D3 | D4 | D5 | D6 | D7 |
| Control group | 100 | 80 | 50 | 45 | 20 | 15 |
| (not administered with | | | | | | |
| bacteriophage suspension) | | | | | | |
| Experimental group | 100 | 85 | 85 | 80 | 80 | 80 |
| (administered with | | | | | | |
| bacteriophage suspension | | | | | | |
| through intraperitoneal | | | | | | |
| injection) | | | | | | |

As is apparent from the above results, it can be concluded that the bacteriophage Bac-FRP-5 of the present invention is very effective in the treatment of diseases caused by ETBF.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

### Accession Number

Name of Depositary Authority: Korean Collection for Type Cultures (KCTC)

Accession number: KCTC 14403BP

Accession date: Dec. 9, 2020

1-16. (canceled)

17. A method for preventing or treating an infection or disease caused by enterotoxigenic Bacteroides fragilis comprising:
administering to a subject a composition containing Siphoviridae bacteriophage Bac-FRP-5 and a pharmaceutically acceptable carrier,
wherein the Siphoviridae bacteriophage Bac-FRP-5 is deposited in the Korean Collection for Type Culture (KCTC) under accession number: KCTC 14403BP and has an ability to lyse the enterotoxigenic Bacteroides fragilis cells and the genome sequence as set forth in SEQ ID NO: 1,
wherein the Siphoviridae bacteriophage Bac-FRP-5 has a latent period of 10-100 minutes and a burst size of 150-540 plaque-forming units (PFU)/infected cell,
wherein the Siphoviridae bacteriophage Bac-FRP-5 has structural proteins in the size of approximately 42 kDa, 48 kDa, 63 kDa, 65 kDa, 75 kDa, 88 kDa, and 118 kDa; and
wherein the composition has a concentration of Siphoviridae bacteriophage Bac-FRP-5 of 1×10⁴ pfu/ml to 1×10¹⁵ pfu/ml or 1×10⁴ pfu/g to 1×10¹⁵ pfu/g.

18. The method of claim 17, wherein the pharmaceutically acceptable carrier is lactose, dextrose, sucrose, sorbitol, mannitol, starch, acacia rubber, calcium phosphate, alginate, gelatin, calcium silicate, microcrystalline cellulose, polyvinylpyrrolidone, cellulose, water, methylcellulose, methylhydroxybenzoate, propylhydroxybenzoate, talc, magnesium stearate, or mineral oil.

19. The method of claim 17, wherein the composition further containing one or more selected from the group consisting of a lubricant, a wetting agent, a sweetener, a flavor, an emulsifier, a suspending agent, and a preservative.

20. The method of claim 17, wherein the infection or disease is acute or chronic intestinal disease, selected from the group consisting of diarrhea, colitis and colonic neoplasia, bacteremia, and colorectal cancer.

21. The method of claim 17, wherein the composition is a solution, suspension, emulsion in oil, water-soluble medium, extract, powder, granule, tablet, or capsule.


SEQUENCE LISTING (1 sequence)

SEQ ID NO: 1 - DNA, 46506 bp, unidentified
  misc_feature 1..46506: note = Siphoviridae bacteriophage
  source 1..46506: mol_type = unassigned DNA
ggtgagtattatattctggttcgacacccggacaattccaggtgctgaagggtataacgttgagttcgtcaagcaacgtatattacgagcgttaacgcatgaacttgaattgtcatacggaggtttgaacgttaagcggatatttaatgacgcaaagaatgtgtatgacggttttagcatagacaagaccgacaatcaattctacgtatatccttatgcttgtctaaggcttgtatgtgacatggtagcaccggaggcgtgttatccataatacgataaggggaatatatatatgtatatatccccttttgtgttaaataagtgttaaagattaaagttttgctttatgtttcaaatatgaatactatctttgttgtgtcgaaagagatagagtagtaacaataaaaaatgaaagatatgacagcaacagaagtaaaatcagtattagcagataacagaaatttagttattagcttttttaatgaaaatgttaagcaagataatttttataccttatcttggtttatgacaaggatacttttggaggctaccgcatcatggggaagaagaaagaacgtatctgaaaaagaagttatgcaggttatcggtaaggttatgcgtacttacccccaaatagcaaaaggacacgttagcaactatcaaaaagcagttaaatacttcggtgcagaacttgcaaatcaaatatctaatgcaagatagttaaattcatgttaaatattaaagttttgctttgcattttaaaagttatgcttatatttgcaatgtcaaacaacgaaagaccccacaatctaaccaagacgcaaaaagattgttgaaagatcaagttcgtaagcgtagaaaataagcaacggtatctacaaagggttaaatgaaggttcggtatccgattaaatgaagctataaagcctaaatctttcgatgaatgacaaagtagtaacaattaaaaaacaacaattatgaaaaaagatttatttattcagagaacagttgaaaaatttattatgattgaatttgttaagggcaacatggatactaaagagcaggtagatagtatgatagaattagttcaaagaaaattagacttttcatataacgaagcatgtgactttataaagaatgctatcggtataaacgcttaaattaaaatattaacaagtgggggtaataccccacatttaaaataaaaataaaatgaacgaattaatttctattagagaaaacaaagggaaacaagttgtttcagcacgtgaattgtacttatcgcttggttacgatagtagtaactattcaaggtgggtaaagtctaatataattgaaaatccttattctattgaaggtgaagattgggccccactcgtcacaaatgacgagcctattaatcagaacgttaacccaacaaaagattatgcaataacaatagttatggctaaaaaaatagcaatgatgtctaaaacagagataggaaataaaattagggactattttatcgaatgtgaaaagaagtcacaactttatatacctaaaacttattcagaggctttaatgttagcagctaaacaagcggaagaaatagagaatcagcaaaagcagattacaagcatgaagcccaaagccgaatatttcgatgaagtgatagatcggaacggactaacaaatttcagagatacagcaaagctgttaggcgtgtatgaaaaagcacttatctttcttttgattgacaagaagtacatataccgagatcagaaaggaaagttgaagccaattgccaagtatgttggagactatttagagttgaaagaatgggcaaaaggtgaaaagacagggacccaaacactggtaactgcaataggtagagatcattttttaaaattaattaataacgtaaaattgtaataacatggagaagcgtagactatctatatcagacaaaggtatgtttattccggaagaaggggaaatattttttgcggaagtaccaaataaggggatagatcgaaaagtgaaaacattggtgtctaaagatagggaagcatgtgagaagtgctgttttcgtagtggtgaattaggctttttgtgtcggggagtacgttgcataacaatagacaatgaaacacaactaacatttaggagggtatcagatgggaaaatttaaaagcgtagaactgtatgacaccttcacaatagatcacccagtgacaggggaaacgatcagagcgcaagcaatggaaggtaacaatgtaatatcatgcagagaatgcctatttagacaaaaggagtttaaaaagatatgcccgcttatgcgatgcgtcgatatggctacgggaaagtgtcaaacctataaacaagtgaagttatgaaaagattagattcatcatgcctaccaataaatttggaggtaggcgaaacaatggagttgatagataaagacggtaagtatcacctattgaagtgtgttacatcagatgaaaatagttcgtgtgaagggtgcttctttactaaacgaaatataccctttaactgtgattctgtaatgtgctcaggactggaaagaggcgtgaaaagaggtgatgtgatatacatagaacttccggtaaaggacgaatttaagcaagacgaagaaagcaaagatgatatttattacgaggaattaacttaaatcaatggttacttttggtaagtatagtacttacttttagtaactgttaaagcgaaagtttaaaaattaaagttttaatataatagtgttgacttatgaaagaagaagttattttaatgctctcagagctacgttctcaaatcaatgacacaattatgcgtgtcaagaaagaaagttccgcagatggcaaggaaatggtgtctatgttgatgtcttgcgacttcgttgatgtgtacgacttgttggtaggagaaacgagcgcatttttggagtatctggaaagttgtggtttcatcagaacggaaatgaaaaagattaatgttaatagcaaccgttcttatattcagagtgaaagggtgtctattcacgaatgggtgaagtttgtgaagggtaaggggtgtttgtgtcaccctgcattgattgctttgtataggaactatacagaaaatagctaatcatgaattattaagtaaggaataggaggttagcgcctccttttcttgtttatatacatttgctttttatccgccctcggagagatttagactaatgttttaattatcaattgattttgtttttcgctatttattcatacatttgtacaaaactaatatgttagaatatggaaaaagtaaatttactattaaattgtgcgctgttggtggcgtttatggcggcttttgtcgttggtttattgcgaaagtggggagtaatcgaaagattgcaggtattcggtgatatgtggcttaaaaagctattccctgcatatagtcgtagtttcatgtatcagttggcaggctgcaacttctgcctgtcgttttgggcttcgtttgtgttatctatgatgttcgctattacattcggtgagccgctattcctcgctacaccgttatttgctgcgcctgtttgtcgaatcttaatttaatcagtatgaagtataatagcgtattaccaccgtttgccacggttgagattatcccgtatgatggcatagagtctttgcgaagtcttaccgataaagttgatatgctttcagagtctgaaaacatgggagtatacgaattgggcgaaagaattatgaaatgttataaaggtgattttctgattaaagaagcaaacgggcgtatctattgcctcgactctgatgcgacgtcaattctatttgagaaaggaggtgaggaatgaaagtaggacggcatgaagttgagttgtacgaagggatagactcactacctattgcgaggtatcaaaagttcaatcggttgatgttggttgattccggtgtaggctctactattgaggaattggatacccatctgaaacgggctattctgtactgcaagactaacccatatcacacgtacaccgaactattaaacctcagacagtcgttcaacatggctatgaatggtattcaccctggcatgattgctttcggtgcgttcgttaagtcgttggacgggaaggagtatccggtacacataacggacgagcaattgcaggaaatacacaccattctatcagatgtaaccgtttcggagctatcagaggcaaacgaggcggtcaaaaaaaaaatagagggtgagatgtcggtgtacttcccttcattgagcgatagccctcagatcaaggagtactacgatctgaaattgcagctactaaccgcaatgttagatcaaacagcgaacgggacggatagaggtgaggaaattcagtcattgaccgatcaactgacggtctactacccgcctcgctgctttcaaggtgagaagtcagtggagatacagtcggataaggagtttgcggagatgtgcctgctaatcacaaaggagatgcacctcaatgcaaaggacatgaccgtatttgatttctactcagcattcgagatgattaaaagacaaagtaaaaaagctaaaaagtaaattatatggcaaacgatgttaaaggaataaaatatagcgatctgatacagccagataacagcatatcggaggcggtcaagcagttggagcaactgcaaaagctatatgaaacgatgttaaagcgtatcgaggaaggtgcgaagggactgcaaaagcctctaagcgagggaggcggagcgacggaggaaggacgaaaaaagattgatgcttatgaaaagcaggtgagatcactggcacgtgctgaacttgatttgaagttggcaatgacggataccgctaaggagatagccgtattgaagcagcagcagacggaccaaacacgcctcaataagttagtagctaaacttaataactcaatggcgggtagctacaatgccctgtccgcacaatacgagctaaacaagattaagatgaacaatctttcgcagtcgtatctagagaatacagaagctggaaagagacttgttaagcaaacagctgagatatacgcagcgatggataagtatcaaaagagcaccggaaaacacactctcagcgtgggtaattacaagcaagcgtttgacggtttaggtttctcagtgtcacaggttgcacgggagttgccatcactcgccatcagtgcaaataccttcttcctagctatctctaataacgttccgatggtgatagacgagatacaaaaactgagggctgcaaatgaagcggccgcaaaagcaggtgaggctcaagtaagtataaccgggaaacttattaaggcgatgttttcgtttaacacagttatggtacttgtgcttactgcattctcgatttggggtaaggatataataaactggataggaagcttattcaccggaaagaaaagggtagataacttgacaagcagccttaaacacatggtagacgctatgcaaaacgcacgcttagagacagcgaaagagactttaaaactcaacttgctgtacaaaacagctacaaacaacgctaaatctacaaccgaaaggacgaaagcggttaaggcattgaaaaaggagtatcccgaatactttaaaaacctaagcgatgaagaaataaagttagggaaagcgtctaaagcatataaagaagcaactaaggctattacagaaaatgcaaaagcacgtgccgcactggataagattacagagttgcaaaaggagtatatcgaattagaccaaaaacgtatcggcgtgttgacgaagcaagtgcaggcacaaggagaacttgcaaaagcggagaagtacaccgcaaaggtatcgtctaccataacagcaacgtctacacaggcagcgagtcaatattatgcagcgacggcaagcaatgttaataaactcaaagatgatattaaggagtacggagatgaagctgaaaagacagccttaaggcaaaaggtactagctaaatcaatggaaaaccttactaagtatgttaccgttgattctctgactggtaaggacacaaatacggatagcaaaaagaaagagttcgacttactaaaagcctacgaggaaagcagagtagctctgattacagactcccgaaagaaagaagaagcggagataagagaggcagctagggcggaacttgctaagttggagaaggacacaacggagaagcaaagagccacacagcagtatgcagataccgtttataacatagaggcaaaactgcgtagggacttggaaaagttgcgtgaaaaatgggacttagaggacttgcagaaagcacatgactttatgaacgaacgtctgagggctgtgcgtgccggaactggtgaagaactattacttcaaacataccttcttgagaacgagcgcaaacaggacgaactgaaaattaagcagtcaacggacaccgaggctgtaaagaacgagcgtcttttgatattgcagcgtgcgtatcaattggcgtctatcaagctacgggaggacttcacgaaggatcagaataatcgtataatatcccggtctgtgttccgcttgcagcaagaacaacaagcgagtgaatccgaatttaacattgtgcagcgatcggcaaaagagcaggaagttttccggttgaatgcagagcggcagaaatgggaacaaatattagagctaacacgattgtacgggtctcagattacaggatacgaggttaaaacggttgaggacactatcgcaggtatagataatgccatcaagcaaaaagcgtccggttgggattctgaacaaggtgtatttggtaacctgtttgatctgatgttcggaggtgcgttcggtgacaagggcggaaagtccggcaaagagcgttcagaggaatttaagcagtcgattgcagacgcttcggagtacgcaatagagaatcttaagagcgtagcacaggcaagggttgaagcggcagaaaaggccgtgcaagctgccgaaaaggaagtatcagccagacaaaaggtgttagacgctgagatacaagcgagggcgaacgggtatgccaacaacgtagcaactgcacaaaaggaactggactttgcacgcaaacagcaagagaaagctctgagggataagaagaaagcacagaaacagcaggaacgcatagatacgcttatgcaggcaagttctttggtgactgcgaccgctaacctgtggaaagatttaggacttgctgctattcctgcaattgctcttatgtggggctcttttgcttttgctaagatcaaagcctctcagttatctaaagcctcagaccaaacggaggaatacggtgacggtacggtagaaatgattgattacggaggctcacatgcttcgggcaatgacgttgatttaggcacaacaaaggacggtaaacgcaggcgggtagagcgtggtgaatatttcgctgtaattaacaaacgttcatctcaacgttatagacgcttagttccggacttgattaattcactgaataaaggtacttttgaacagaaatatctaaacgcctattccggtgctgatgaagtcacgaacattatgcagggcggaaacgtagatttgtctaatgtcgaacgtgatttgaagtctatcaaggagagtgccggacacaagtttataaccggagctgacggtacaatcattgaagtgaaaggaaatgttaaacgtataatcaaaacgaaatgaatgtaaaggacttgaggtttaaaatcgggggtgtaattgtacaccctctttacaccgagctaaaacggaagttcggcaaagagaatcaacaggagtttttcagagagacaatagaaggtagtttaactttcatcggtgcggactatttgttaataaagaataagagcattgaggatgttatctacatgactatcgaacaaaaggacaaaggacagacggaggcgcaatatactgtcatttatgaagcctatttcagtaagactgattgcgagatagataacgacaatcggagttgcaaggttaagttatcacctaaagacgcttattccggcataatgaagaacattgagaataagtatgacttaattaaactatcgcctgcattaacgcagatcggagtgtataaacgtccacttgttcaggtgtatattgcgggcgccggaacaatatctaactaccttgcaggaactcagtatgaaactgatgtgtataacgtagttacggatagtaaagagcttactgacaaacactactttgctttctttgctgcatataatgaagtagaagttaaggcagttccatatcaatcttttaacggcaaatactacggaaagaacggtgtatacacgaagttggacgggaattactcaattaaatggagttatagcgagggtcttaatatcggttatctatcacttgaaaatagggacggtgtaatactgtacagatcgcaaaaaatcacatggaaggataagagctatttctacatagatgtttctgaaattacgtttgaaagggtagtaaacgaacctacactccctcagtccttcggtggaaactcggtattgcttcaaaaggtatttcaacggttgctgttagaccttcctgagtttgacggtaaaccaacagggagattatcttccgaagatgtttacccgacaaatagtaattacctgtatgccgcacctcttgtgggaaactatttctatacgtctactaaggtgcaggatgttcccaccgaatatggcgtaaacgatgaaggaaaatactttgttgataactttctccctgccgttgttggagcgggtaaactgtatccggtgtgtcggtcaaggtgggggaatatgtctatatggttcgagtacgatttagggtatagcgcactggaagaacgcgcccgaaagaagtacattcttaagcactcctttgccatcagtgacgtaataaagacgctactcacacaggtagacccaacgttgcaccacgaagccacagaggaatattcacgtttcttatacggtacgtctaatccgttgactggcgcaccttacagagtgtttatcactccaaagagtaacattctaaagggtgaatacgatcagcccgctaagaaggcagaaacgacactcagcgacatatttaaaatgcttcgtgacacaatgagactgtactggtttatagacggtgataaactgaggatagagcatatatcatacttcatgtctggaggctcttatactggtactggtacggtcggcatagacttaactaagttgaggtatgcaaagagtgggcaactattcacgtttaagaccaacacgcttaagtatgataaaacagacctgccgtcacgctttgaattttcttggatggacgacacaacgaataccttcgcaggcttccctattgatgttaaatcaaactatgtgcaggaagggaagaaagaggatattagagtagctaacttttcctcagacgtggactacatgctgctatcacctggtgacttttcctcagatggttttgcgctgttaggtgcggttcaaaagtcaggcaaatgggaattaccttttgttactgtgccgctgacggataaatcgggtaacaactacaccgttacgcctcaaaacggttacatgtcattcctacacctcgtgaagtactacatgtacgatatgccagccgcaaacattgagcacgaaggagataaaacggtgaccgtacaacggctgaggcgaagtatgacacaagacttatcattcacctatgacacaacgcccgatccgatcaagctaatgactacggacgtcggaaatgggaagcctctaaccatgactgaggacttaacaactcgtgaaataaccgtttcactcacatataccccctcttaatgggggtatttttgtatatttgcccaataatcaaatttttataaatatgaatacattcaacaactttagtccgttagctttcagaaagaaaagtcagaaagctacgtataaaaaatggtacgcctatggtaaggagttcgctttgccgtttagtacaaccgaattgccaccgtttcagtttacggttaccaatcttccatcatttgaccctactacggtagaggtgtttcttgtgaatgaagctaccgaagcgagaatcggaacgggtatcaaaataaaagttgatacgatggacgaacataactcagttttatacgtatcaccagggagcaatgtgtatgctaaatcagtagagccgggtgtatatcgtgctgagtttactatacccgaaggtgaaacatacatatccacgcctatttgcgtgactgatggaattgaaactaataccaactttgttaaattggagtactggaatgatgaaaagttagcttatcctaatggcttcgttaccactggtacggataatgatttcaagtttcagatgtatatcccgactaccttctttaagccgaaatacgagtttgaggaagagataacaaagagagcgggctataagtttctcgaattgcagacttgcaacaaggtgttcggctttagtttcctcgcacctgagtatatctgtgacgctctgagactggtgcgtctgtctgactatatccgtttcgcccatgacggtgagtattacaacgctctgaacttcgagtacaatccggactggcaggataacggatacttggctgctatcgaatgccaatttgaaacagacacaatcatacaaaaactcccttctttcaatcggagagatagagagtctttttataatgccctactggcggatattgaaactccaattctgtttagtccggacacagtagggctgtattaccgtgaatttaaacagggagagccgacaatcaaaggtaagttaatccgggaactatcacctattgacttgatagacgaaaatactactattgccgtggatatgggtgctggtgaggcgcgtaaattcaacctttaccgcatgttagaggggtacatctcgaagaatcacgaagatgtaacggaattccttttgtcccttcgtggaggcgtgaatatcggtacacctaatacaagcggtgagtatcctgcaagcgtagacagggacgggaacactaagttaaaggacatacaggggaatgatgcaacgttgaacaacgtcacaggaaaagatgctacgtttaaaactgtggaaactggtttcttaactgttaacaagactgccgcaacaattgacggaatgggtaatgccaatgtaaacgatttaactgcaagaggtgactctaagttgcgcagcgacgtatatacaggggcaaaaaatggcagccataccggaaagataacgaaagaaggacagttgcagtacctttcagctatcatctacgagttcctttcgtccgaaacgttcgttcctggcttcttaggtgaaggatttaaaatatggttggaaagcggtaactggcatatcgaatgcgataacctaaccgtccgccaaactatgaacatctttgagttgcttatccaaaagatacgtagcgtcaacggagctattgttgtatcccaatcaaacggtaaattgtcatccgttgaagaagtaggaacacaatacaagctaaccacaggcgatgaatttcccacttttcaagagggcgatttggtccggtgccagacgtttgcaggctatcaaggtgcgggatttacttttgactttactcagtttgcaaaatatgactattccggtggtgcttttgatagcagcttgattgatgttacacccgactctattagctttaacttgaatgatactggtaattccggttttgcattctataaattttcagagtcaagccctacaccaattgaaataccttcatttaccttgactttggagggaggctatcctggtatgatggcttttgctgccggacttgattcaaatgacagtccggtagagggtgtaggcgtattgcttcaaaacggtgataatgttattccggctattaaagcagcgcaagacatacacaactttgctataacaataactggtgattccggtcacggtgatggtaaagttacagtaaagcaaaagaaagcggcaggaagcgcaccaaacaatagcttagttaaattctattgggtggaagttaaatcggttgacggggcttctttctttgcagataaagcggagttcaacggtgttgttccggctgtcggtgacgaagttgtgcagatgggtaacacgaagaactcagaaagacaggcattaatatacatcacagcgcaggaaagcggcaagccgtacattgagattctgaacggtgtcaaggcaaagagcctaaccggaaccgatcgtacccgtttgggcgatttgtctaacattgtagaccccgatttcacaggtgatgcggccgtgaaaggaactggtttctattctacgaatgctttcttaaaaggtatctttgtacttcgtaacggaaagcgtgtagaggacgaaattaagatcgcaaaggacgcagccgatcaagcagcacaggacgcagcgaacgcagcacaatcggcacaggaggcgaaagatagacttaacaaatgggctgatgatggctttatctcaccgactgagaaacctgcattgattgacgaaggaaagcgcattcaagccgagtatctgcaaattaaagcgaatgcggacaaatacggtattcttgtaactgaatacacagaggcgtataacaactatctgaacgaactacgctatcactcggccgctacaccggaagatattgccgtgcgtccggaactggcacagagtcaaacggcttactacgacaaacgcaacggagcgttgaatgccattgcggacgctgctaaatcgtatgtagatgaagcggataagaagctaaaggaatacttagatactgagataactgctattcccggtaagattgaactcgctgtgcgtagtttaaaggtggctgatgttaacttattgaaaggttcatattatgagctgaaaaatgatgcctataacgtggggtattaccattatgatgtaccagctatagacggaaaggagtacactttaaccgtatgttacacacttggaagcagcaatacagaaataatagcatactctaatggtggcgtaagtcgtattgctacccttggaacaaaaggagataaagttgtagaaagttataagattactatggcaggttataaaccgagtgaaggtatgtatttctttcaattcccaaacggtacatacggttctaaagtacattgggctgttttgactgatggaaatttgggtgtaaccagttggataccgtctgcaagcgaaaagaatgtaggacttagaaacctatgttcgtttaagcgtatcactgatgctggctttactttcgcctcagattatcaagatgacggaacatttttcattgatttacctaaacttgcttccgagagttatcaaagttcacaaaaagatatgtttggtctgacttatgacgcaaacaaaaagtattatatattttgtgatacatttagttatataacaccgccagatacagataaaaaaggtacgttaactatatatgtatattatactgatggtacacatgatttagctgtttatttgggtcgagatagagcgtttaacaactgtttcactacaaacaaacctgttaaagctattgttggtacttatggatatggttttcacccacgtgtcagacttggtgtgtacgaaacaaatttccctgtgtcttggagtccagcacccgaagatcaattgtatcaatcggttaaatacactgatacccaaatcttggcggtagatggaaagattgcattatctgttaaaactcagttagagaaatctactatcggaggtaacaatttattagacgggtctaaaggttattggtttaatgaatcagattatttatcaaatgtcgagttggtgcgtgatggtgacgctggaataactcgtattaagggaaacgggtttttcaattcatataaaagatggatgccagttgataaatcagagatacgtaatggtaaggaatacaccgtaggtatagatataaacgttcaaggaaattattataatgacggttatttttgggtaaatatacgtaggtatgaagattctcaggagataatgttagcacatgaacaggtttatattgaaaataaacccaattgggtaaggtattacgttactgttaaaactcccgatagcggtaatccgaacgattggctattcctttgtggtttttccggtaagaacgaaacgaataatgtaggtgtgagttattattatagaaatataaatatgactgatggaagtatcggtgcttcgtggagtccatctgccaacgacacctacaacaaaagcgttgaatatacaactcagcaaataagcattgttgagggtaaaatatcacttgaagtcagcaaagagatacaaaatactcaattcggaggtaacaacttgtatagctacacgagttcaccgcttaatcagatgagtcctgagccaatgactgtaataaggcttataagtgaacacggatttcgtttggtgggtggaaaaggcggtcagtcccatgtaagaatacctaatattataccgcctatccccggaaggtatactgtttccggatggataaaagggtctcaaagtacaacgcctggtataactattgatgtatgcgattctgaaaggattcgtataaccgctactgctgacaataattggagttattttaagcacacttttgatgtaagcaaaaatgcaagttcaccggaagtgtataattttgttgatatagaagaaatatcatgggcttatatatgggtgaaagatttcaaagtagagtttggcgaagtcgcaaccgcatggagtcccaacgaacaagacccagtatataaatcggctgagtacacaaacagtaagattgacgtaacaaatcagtctattacagcagcagtacagcgaataaacgcaaccgaaaacaggttaacgagtgccgaacttaaactaactgatacgagcgcaaaactatccgttgttgaaggtaaagcggatggcgcacagtcaaccgccaatagtgccaacagcgcagcaggcgcagcacagtctactgcaaacgctgcaaattcattggcaggcactgccaacaataaagcggacgcaacaaataacggtttggtcaagactggaataaacatcacgtcccgcaaaatcgtgctaaagtctgataacgtcctctttcagaataacgcaggacagcaaacggcagcactgaacgccaacggtcggttgagtgccaacgtgattgaggcgggtgaggttgttgcgaatggctttgctgcacagcgaataacaaccggaaacttgactgtgacagatggtgcggtgattgcgggaatgactatttctggaggtgtcctaactggtaagaatattaatattacagatggcgcaaaagtaggtagttttactatctcgagcggtatattttccgcacaagaagttccggcaggtatacaaatgactctagcgggcaatgccgctacttttgatagtagtggagtacgcgtagagaataattcgggcagctatgcgttgactactacgggcaaaggaaaaatattcttaacaggttcggatttttgggtccagtgtaaggacgttgattttatgggtgctcaaacatggaaagccccaggtgttttttacgcatgtacgattttgggaaacggagcaattggtaaaacatgggggaaccctgactttcacataacaagggtaattaaaaactcaacagggagatatactgtttatacgactggttcgaatggagattactttgttatgattacagcgtataatgttacttcatggctcagtacaacagtagaaccatactccgaagggcagtttacgtacaaagtattcgatgtaaataatggcatgactgacagcgcagttattatttatttttgtggcatggttaggtagtttagtgttttaattgacggtaagttggttttatccttcttaccgtttacctttgtagcaaataatttattcattcacaattaaatatcaatttttatggcaacaaaagaagctatttttagtttggagagtgttaagtactcgaaagaaacacagattttggattatagttttgagactgataacggactttttaaaggtcagattacaatcgtacagcaacccgatcaagtaaagcagatcacacactgtacagctgaggtatcagttaaagaaatggttcaagtgccgggaacggataacacgcctaccatgcaggaacaatatgttaagttgggcactttgtcgatgtcgcaggctcgttttgaacttaatcaattccctgtacacgaaaagacacccgctttgttgggagattttcagaactacatttttgcattaactaaacaatcagaataatgtcacaggaacaaattaggctgctgatggtatccacatgcagccctattcttgcatttttaacacctacgtccggattcttgacggcacttgtgtttatgtttgtctttaacataatttgcggtatgcgtgcggacggtgtaagcgtgtcaattagaggagctcaaagattcactatctttaaattcgtatcggctttacaggagtttttgctgtatatgatgattatagtagtgatattctcggcagttacaaagatgggagacaaagacgctgcaatcatgtgcgcaaagacaatcacgtatgtgtttatgtacgtctatttatgcaacggtttccgtaacttgtgcatgacatacccgaacaacaaaggtttcaagctgatctatcacattattagatttgagtttaagaggctgatgggtgaacatgcgtcaaagataatcgaagagcaagaagagaaagaaaaacaggttattcacaagggggtataatgccccctttaaacatttagattatgaagtattttacgattaaagaacttacaaagtcctctacggctgaggctaaggggatagataacaccccaacacaggaggttgagcgtaatttaacggctttggttgaaaacgtattagaccccttgagagagatttacggtaaacctataacggtcaattcgggctatcggtgcccggagctaaacagggctgtcggaggcgtatctaacagccaccatgttaagggatatgctgctgatattacagcgggtagtccggaagaaaacgaacggttgtttaatatcattcggcacaactttaaattcactcagaatttagatgaaaaaggtttccgatgggtgcatgtgtcttacatcccatctgatttgcgttgtgagtcacttcatttaaaataaaacagtatgaaagctaaaataacagccatagtagcattttctttcctttgcctactaattgtataccttcttaggtataacgcgaaactgagggaagaaaacggcattctgaacaggaatgtaagtgtacttactactcagaatgtagcataccgaacggagtccggcaaatcagcaatgaaagcggaggaactaaacctcgccttgcgtcaataccggaacacattgcaggggaaagacggcactataaaggacttgaaacaaagtatcaaagacttaaacagtcatacaagcattcaaacgtcaacggagagcgtattttccggctctctacgtgatagtattataattcgtgatagtttggttgctgacacattaaaatgcttgaatttcgcctctaaatgggtaaatgttaaaggatgcatagaatcgcccgatgtatttgcgggcaaggtaaccgttcgggatagcttagagttattgaacatagaacaccggaagcgtttcttatggtggagattaaagaaggtgaagtatagggagtttattgtaaccagtaaaaacccagatacgcaaatacttgatttaaaggtgactacaataattaaatagttaacgttggttaaagctattgcaggtataaaaataatgcctacatttgcactcagagaattacaaataaataaattattaatcatttctttattggtaagtctttatgtagaagcaaacgtatcattaacaaagcgttgttaataattgcaatcatggttaatattaagtgattattccctactggtttgtgaaaataggtaggtttttaaaaagagatttctattcataaatatataatataggttaattagttaggtaattgttgtttattggattttttgtcatttttcatttttccccgttgcttgtgaaagtagcggggttttttattgtcttgtctcagatgtgcaaacgttaaataagtgttaaagattaaagttttgatttgcaatttaaagttttgctttatatttgcagtgtcgaaagaaacaaagtagtaacattaaaatatacgattatgaaatttaaaacatggtattttagagatactcttaaaaaagagtgtttttcagtagaggctaaatcaattagaggcgcattgactcagattaagaaaaagcagcgtgaatgcgctgaaaagttcgggcatacggtattttggactatctttgaaaacgaaatggccgactcaaatatgcgctgcatattggtaacgtatgtgtttcccaatggagatattaaacaagatgtattataagcaaattgaatcattatgaaattcataaagaagcaaagtgataagatactgatagccacaagggacgaagcgtttgatgtatacgtccgcagtcacccagttataagtaggcttattagggagatcgggatagaaattattcagcaagcgtactacggcaatcatatagcccgaataccgattgggacagatgataaggatattttaaatacaatctatcaagtattagagaatgacgggtataaacattccttcgatatagtagaaaaggttttaacagtaaatatattataaacagtaattttaaagttatgaataacattattaaagttggcgaaactataaacgcaaaagagtatatgacatcaaaagagattgcgacaattacaggaaaaccgcataatgatgtattaaaagctatacgggcaatggagaatgcttgggtgaaagttaacgggggaaatttttccctcgttgaatacacggacgcgaaaggtgagaagcgtcctatgtataagctgacaaaaacagagtgcctgtatatagcaacaaagttcaacgatgaagcgagggcaaaactcgttatccggtgggaggaactcgaaacaaaggaaagacaaaatgtacctgctcttcctcagacatatttagaggccttgaaagcattagttttatcagaggaacaaaagcaagttcttgcattggaaaatgaatcaatgaagccaaaagccgattatttcgatactttagtagaaagaggaagtaatttaaatctaagagatacggctaagatgataggagtttctgaacgtttctttatagaatatttgttattgaatggatacttatatagagatgctaaaagaaaattaaaaccgatagccaaatatgtaggtaagtattttgttctaaaggaatgggtcagaggcgaaaatacagggtcccagacattagtgaccgtgaaaggaaaagatagattctataaattaatcaatgagtaatcaattttaaaaattaatcaattatgaaaacttcaaattatttgttcagtgttttagtaggtttgtgtttgatggcaattgccgctttcctttcgtcttgtggcgatgaagaaatgaagtatgaaactaattattcggttaccgttccggagtggcaaacggtgtacgtagagggtgagtgtacaacgaatgtagccttatatgtttgggataaggtagagttaacgtcggactacgttaaggtatattcaatgggacatgtgaattatctgaaagttacctatactgaaatggatatttacggctttatcatttaccacattgatagctataacaaagaaactattcaataccacccgaaagacggtgttcttcgacactctcagcaattaaacggagctgaagtaaccgttgtttttagaccattacactaacattaaaaccttccggcgtacaggtcaaccgggtaaacaacagatgaaagatagaataacagaaaaccaattgcagctaattactaaggataaaaagctgtcggagatgatagagggaatcactcagatacgttctattgtcaaagaaaagatgaaggtatatgaatgtataacatatactgaggacgatataaaagaggcaaaagcagatagggcaatgattaacaaatcgtctaaagccttaaacgatacccgaatcgaactcgaaaagatatacatgcagccctttaacaagttcaaagatgtagtaaaggatacatgcgacatgttagacctcacctcaaacaacatagattcacagataaagaagttcgaacaggaagaaaaggacgcaaagatgaagcaaataagggattattttgacgaacacaacgagtaccttataaattttgaccgttgctttaaatcaaactggctaaacaagaataaaggtattgctattgtccgggccgagattaacgaactgtttgaactggtagcggctgacttcgagaaactgaaagagcattttgagggtgaaccttgctacatggctataattgaccgctatcaagctacgttggactataacaatacctaccaatacggtgtatcactcgttaataaggcactggaagccaccacagcgctaaactcgcctcagacgactaatacacaaccaccgcaacaacaaccgctaaaacagccggaaaacaagcctcagaacgaacaggtttatgtaagagcgtttaaggtcaaagtaacaagggaacaagcatttgcactggctgactttatgttctcaaacaacattgagttcgagagcattaaactataagaggggatatttccctcttattttattccattcacaaatgttaattccatgttaaaacttaaagtttttctttgtgttttaaatttaatctttatatttgcagtgtcaaaaggaaacaaagtactaacaatttaaaaacaaatatcatggttataaaaacaaggtttaatataggcgataaagttctatataaagggaatgaatttacggttatttctattgattgtatgaagaatgaacgaacttttctcatacattatgtgataagacgtgcaggattctacgatagaactattttggaaatggacttaattaaaatgcaagttacacaaaactgaaattagtgatatataattggtaaacatattataattaacaaattatgaagcagtatttagatttactttcttatgttttgaattatggcgaaaagcgagaggatagaaccggaacgggaacaatcagcgttttcggaagtactcaaaaggtttacgatcttcgtgacggcttcccgcttgtcactacaaaaaaggtattcacaaagggtattatccatgaattattgtggtttattaaaggtgataccaataccaagtacctaactgagaatggcgttcacatttgggacgcatgggcggacaaagacggtaatttagggcggatatacggtgcgcaatggcgtgactggcgtgttaatagtcggacaagtatagatcaacttaaatcagtcgttgagatgattaagaaagacccgtcgtcacgtaggttaattgttaattcgtggaacgttggtgaccttgataaaatgaacttacctccctgtcattgcttttatcaattctacgtatcaaagtacggatttttagacttgcaattatatcaacgtagtgcagatttgtttttgggtgttccgttcaacattgcgtcttattcgttgcttcttgctatggtggcgcaagtatgcaatttgaagcctcgtaaattcattcatacgttgggtgacgctcacatatatttgaatcatgttgaacaggtaaggacgcaattagatcgcattccgttgcgattgccggaactggttttgaattcttccgtcacagatctgttcgattttaaatttgaggatatagatattattgattataacagccatccggcaataagaggggaggtagcagtatgaataaagatgaattttataaactctgtctttgtgaaacaaggaaaacagtcagagaggttttgaagaggcaagatgttttaacacttcaaatgttgaaacgtagttttaatacttggtggttatatagtgatatttgtgacgctatcaattataagttatcaggtatgtaatttaaaaggggagtacaacgctatgtattcccctttttcgttctatcctcacggactaaacgagtcaataaacaataagtagtaacaagtatttaagaaaagttctacaaaaatagttctaacagtcgttctgtgcgtatcttttcagtgttttaaatttcattaacaataaaattaaagaaattctttgtgtatttaaagttttgccttatctttgcattgttgaattaagtaagtaacaattaaaagtagtatatatgaatcagaatgaagtacatgtttgtccgtattgcggaggcgatttatacctttggcaatcctatccggtttttagcaaagcatattgcgattacgattgtgagccggaacaagaagaagaaaatatctataaatgcagtgaatgtggaagggaggtaatagatgtcgagtgattttaagattaatgaagccattttaaacgctaagttgaagggcttaaaagtgagtaaaaaagagatagctgaaatgttgtgggaggatacaaagccaaaatcaagaacggttaacatgtcctcactttgcaaccgtaggacacgaaagattaatattgaatgggtgtcaaagatatgtgaggctaccggagtggacgcaaattttctgtttaacataaaaccaaaaagcaatgattaaaaatttatcaaagattcaaaacgagatgaacgttaaaaaaggacgttataataagttcggtgggtattattaccgttcatgtgaggacattttgcagacagcaaaagaagtgtgcgacaaatacggttgttatgtgaatgtaaccgatacaattgagtatattgaaggtagattttacgttaaagcgactgcaaaggttgtggatatagaaaccggagaatcagaaacggcaaccgcatttgctcgtgaagaagaaagcaagaagggaatggacggtgcacagttgaccggagcaacctcaagctacgcacgaaaatacgccctctgtggacttttcgcaattgatgatagtatagataacgattcattaaacggaacgcaggaaaacgaagaaaaagcggcaaataagaagggttctaaatcttcatcaacaagtcgtccggataactcacagagtcaaacagctattttggtaggatacgtcaacgagtgtacaacggttgagcaactaagtagcttgtttaaagctaatcaagcatatcaatcggatagtacatttatgaatgcgctatctaagaaaagagccgaaattgaaaaaaagaagtaattaacaataaaaattaataagtatgaaagatattataacattacccaaacttaatcaatcagatgttatctacattgaagaaacacatgagtatttcacaccggactttaaaaaattgcatggtataaccggatttatcaacgagcaattgtttcccggtaaacttgatggtattcccgaaagcgttttagcccttgccactgagcgagggaaacgagtgcatgaagaatgtgaaaatatagataatgaaggaattgaagccgaatcaaaacaaggtgaaaactatctcagattgaaaggtgacttcggactaactcacatcgcctcagaatatatagtaacagataacgagtttattgcatcaccgattgataaggtgtatttaggcagatactccgactccgttatattaggagacataaagactacatacaaattagacatgttgtatttgtcttggcaattgtcaatctatgcctattattttgaaagacaaaatccccatttaaaagtagatactttgttggctatatggttgagaggtgaggatacagatggtattgtgcaggttgagcggataccggacaaagagatagagatatttcttcaatgctgcaaagaggaattgaaatacgtagataattgtagtgctgattcatacgttactaaattaaacgatcttcctgcaaaggttagcaacgttgaggaatcagtatatcagttaatcgaaatgcaaaagacgcttgacgagcaaatgaataagtttaaatttcagttactcgaaatgatgaaagaggcgaaagcatataatatcaaaggtgacttgattacaatcacacgaaagaaagcatatagcctagaatcgcttgatactaaagcgttgaaagagaagtatccggatatttacgaagaatttattaaatactcagacgtgaaagaatccatccaaattaaagctaaataattatggttatagatgaaaagatagcgaacgagatcggcttagaggctgctgccgtttattcggaaatgattcttatcctctgcacaggaatgtacagagaaaagttcaaagggtgtcgagtaaagcaagtgcctaacactgtttttgtttcgatagcaaagctaaaagagatcattccgttcatgtctacgaagaagttgtataacgctgtaaatcgcctcgtatcggctggatacataagagaggcaaattacaggttacccggcatgaatacgactaaatgttatcagatggtagatagatagacccggttctgattgtgatatgcaccccacttgcaagcgttgtgagtggggtgcttttttgtaaattactgatattcacattggttacaatagtgccgaaaattcagctatattcactaataaatggggttactattttggaaacccctactcaaaaacataaaagggatattagtttaactaataccctttgggtctgcctatttttcggcatacccatcaaatttgagttgctctaattatcagacggttgcgaataattaaatgttaaagtcaaaaacaataatgttttttgtttgcaaccaaaactattttatcatatctttgtggagtcaaatcaaaaaacaatatacaacatgaatttagaaaatctttttaaaattaaaaatgtcgctgattatatgcgttgtagcccaacttgggtaatgaagttaattaaagcgggtaaattggaacatgtcaaaattgacggtgcgtatttcgttgttcttacaggtgaagaacttgaaaaatacaaggagtttagaaaggagcttgacgagttgttaagcaaataataattaataattaaaatttcaaattatggaatctaatttgtctaaagttggcgaaactataaacgcaaaagagtatatgacttcaaaagagattgccgaaattacaggtaaacgacatgcagacgtaatggaatctattagagttatggagattgcttgggtaaaaataggtcgacggaaatttccgttaacctcttatatagatcaatggaatagagaacaacctatgtataagctaaataaaacagaatgtctgtatatagcaacaaagttcaacgatgaagcgagggcaaaactcgttatccggtgggaagaactggaaagagctaacagtatggggaactttaacgttcctaaatcattccgtgaagccttgttacttgctgccgaacaacaagaagttattgaaaaccaacaaaagcagattgaggaaaagaatgcaaagattgaggctgataaaccgaaagttctattcagcgaagcggttgaagcgtctaaaaagtctattcttatccgtgaacttgcaaagataatcactcaaaacggttatcagatcggagaaaagcaactgtatgaacgcctcagaaaagcgggctatctttgcagcgtaggagagtcacgcaatcagccgtctcaagcatatatgaacatgggtctgtttgagattaagaaacgggtgatagtaactggtgaggaatctaaggtttgtacaactacggttttaactccaaaaggtgtgaaatatttcataaataaatttataggaaaatgaagaatgatctaacaggtatgaaatttggaagacttaccgctattagtgttagcggaaaggataaatggggtaattttatttggagatgtaaatgtgattgcggtaactataaaaacgttcgtgcatataaacttaaaaatgggaacacgaaaagttgcggttgttattttatagagtcgctttcagaaagatcaaagaagcatggtttgttatccggaggtaaaccaagaatttttaatacttggaatgatatgaaagcaagatgttttaacaaaaatgaaatagcatataagtattatggtggcagaggaataacggtttgtgatgaatggcttgtttttgaaaatttctataaatgggctatgttaaacggttatagtgatgaattaaccatagataggatagataataataaggggtattatccggataattgtagatttgtgactttgcatgataattcaataaggcagagaaaatcaataatgataaaaatagggaatactgaagaatctgcaagcggatgggcgagaataacaggtttatcaagaactgttatagtcaatagatacagaaagtatggatatgaaaaattcaaaaaagctgtcatatcttacattcaagaaccaattggatacaagcattttgataatttattaaataacatggcagggtaatacccgcctataaaaacaaataattatggcattaagtaataaaaactatatcaatatacaggcttggatggttaaggagttgaatctaaccggaaatgatctgatatgctacgcattgatatatggttttagtcaagacgggaaaagtgagttcaacggctccttgtcttatatagctgagtggctaaatacgagcagacagaccgcaagacttattgttaaacgtcttgtagataatggagttatagagaagcgtgacactgttataaatggagttaagttttgcagatatgtagccataaatacggggtgtggtgaaaatcgccacactgtaatgaataacaatacggggtgtggtgaaaatcgccacaggggtgtggtggaaatcgccactaataatatattagataatataaataataacactaaatctactaacgtagattatagtgtagccaagcgcgaagaaacggatttattcgaagttgaatcaaataacgatcctataccctccgagatatttggattcacagctaaaggacttgacgtaacaaagaaaacaatcgaaaggacggataatctatttactcagctaacatttcctttcgagtccgaggactttaaacgcttattctacgttctaatgactcaacctaaatggcgtgtaaagacaaagactctaacagctatgcaagctaacttaaacgagatagcgcaatttgaagaagagtttgctaaaagccttattcaacaaagtatatcgaaagggtgggcgtcacttgtgtatgaatcaacgccaaaacaatatatgcaatggctgagagagaaaacgggagctactaaccaatatcagcaaaacaactctcagcaatataagacaaagcagtattttgctaatgacgagcaccgggagatatacgagagatacctaacagagacgtttgattaacgagaatggcatttaccttgcgaatttaagactttcataataaaaacgagtaatataacatggaaatagagaaatatcaaaatagaggcggaaaagtggctttattgggtggcgtacttccgtcattcgtagagagaaacagagaattaatacagtctaataaaataaagcagctttctaaggtggatcaacgtatatttgtagaatctaccaaacgtttaatttcggaagaagaaagcgaagaaaagaaaatagagtatttaggcattatctttatcggggtatgttctgattttggtctgaatgcaccggaacgtagtgcggttaaaagcgtattttcctcgatttttgacgttattgatttgtattttgacgatctttcattcgcagaggttaaacttgcttggcggttgcttgctgtcggggaacttgataactacctacctaaagatcgctacgggaatccggataaaaaccactacggtagcttaaatgtcgattacgtaacgaaggtattgaaagcataccgaaagcgtaaagctgacatgatggcaaaaactactgcgctatttcctgataagccgaaagcgactccggaacaagaaagagcgtttttaaacgtacaggctaacaactttattttcgctattatgaagtataaatatagcggtcgctttaaggctgaatctgataggcttataagcgagtcaacgttcaagtatatggaacggttggggtatgacatggatactataccgacatacgaggataagaagttagctttagcgcaatttaaaggaagaccgataaacagctttgcacaagtgtttgaaaaggaatgtttagcgactttcggaattgaacatgaagccgtttattttcgggctttaatgatagcaaagaaacgtttgttattccggtactgggacgaaatgctgatagaggaagatagcataaaagatttgtattactataaacattaaaaacatggaaattaatattttagttggaattgaccccggtgtatcagccgggggaatagctatttataaacctggaagcccgcttgtaacggttaaaatgccggaggatcctttggatatatacaggctattcaagaagattaagcgttccggtagcccgatgattgttgttgagaggctgtcaattaggggtgatgataccggaggtaagcaatatcgcatagttactatgcttgaaaattacaacaaccttgtatgctgtgctaaagttctcgaaatacctttagtgctcgttactccgatgacgtggcaaactggtttaggcctgagggtgaagggggcgaaagaggataagtcgctgagaaaagagaagtattttcagtttgcggcacattcatttccgactggtaacgtttttaagtggaatagtgatgcggtttgcatacttcgctttactcagttgatgatagctaacaagcctaggtggatatcggagcatttagccaacaactcagattacgtgttttcttttgatttaacgaacgattccattgaatatgatagattactcagagatgaaaggaaagttgaatatcggtcaaataaatagcaaaataggcgatgttttgattcaagctgtcataagaatgcgagggaagcaaaagagatacgaaagattcggtgaaaagtacagggaagcgaaagaggcggaagaaaagaaagtagatgatattatagcaaagttgaccgactcacaaggcagcttattctaaacaaaggttaaataacgggtatttcggaaagatttacccgttattatttgcgtataattaaagttttgatttaatttgcaatatcaaaattaatcatagtagtaacgatttaaaaacttattaaagtatgaatactaacgaaatgtcagttgctgatgtaattaaatcaccggagtttaaagatgaattaacaaatcagctttgtactatgcgttcagatgttgaacgagctaaaaacaaaattctgagaaatggaggattgacgaagcggattatgttagatcgcattgacgatatgactgtttcggacattatcgaagaatttgagaaaattctactgagaaagagcgatctccctgctgccgttcgtggttttatttcttctttgtgtggtagtgtatttgctaaggtattttctaaaatgaaacaaaatgaagcaaaacaggataacaataccgggaaaagtaacgagtaacggacagttgcagatgtacatgggtgagttaaacgagtttgcgaaactgcacaagggaaagaatatcatagcaagttttagcgtttacgagccttcgcaatcggttgccatgaaagcgtattactacaaagttgtagtaccacaatttcaaaaaggaatgtatgacaatggtaatagatggagcgaaaaagacaccgaattgtacatgcggaacttgtgtccggtaacaatgggtgaggtcgttgatattgatacgggtgagtatcgaagtgacccggtaagtatcaacgatttatcgaacagtgagtttgtagagtatattgattttttaaaacagtttgcagccgaagaacttggagtttttatagaggatgcaacaaaatacgtaaagaaatgaaagaagttagggtagaagattgcgaaatgacgttgagagaaaaattcgacttgatgtgcgaggctctttcggtgtcaccggatgctattttaagccgggagattacgagagatatttcaattagaagaaactgtattattcatcagttgtatgattatcgtttggacggtttgccggagctattggataggacgagggctttaatagtgatagcgcaccgcaaatttcaaaaccaattagaggttaacgatccgttggctattgagtataagcgattaattgatgaaagattggagagttatttgaatggcgaagaagaataaacaaaacttagtcctcgttcattgtacagaatgtaaatatagctcagaccatcacaatttgatatgctattgcagcaaaagaaaacaaaagttatgcagttgcccgaatatcggaagggtatgcgaacattataaacctaaaaacgaatattagtatgttgtacgacaattttgaattaaagagagtaaagttcattccaaacggtttggaagtagattacaatgactgtatgaatgttgacggtgaaacggttaaaacgtttcataaagtgaagaatccggagtaccctcaccccgatttgcagaacgaagccgggaaactgagagggtatatcgtctggcttatgggtctgatgaattttgcaaaaatcacctacttatccgatctttcaaaactggataaggagttggataagcagtttcaagacttctttgaaatacagtcgactcgaattatgattagagagattgtgagagatgaagaaaagaacacagtaattatcaaatacgagttttcaggtacagacttgtcgctgtttaaaatgcagactcctaaaatcaatttggagggtgaaatgcttcaatttgaaattgacatgaatactgacttagagggtatgaagcacgaaatatttgattatctgtttaagggtaagcgtgcacaactttcaatgttcggtgagattgcggaggcggacgatattaaagacgctgatgacgatacagagggtgattccttttttgacgaagaaactgaaagcgatgtatctgctgagtagtccggaagaaatagaatattgcttaagtaggggatataatcccctgcttttcaatcgatatttcgatatagaccctaaagcaaggtatcagtatttgaaaagtctattcggtgactgtcacgatcagagggcaaacgagcggttttttcggtatatgtgggagattaagcctcactattgcgaagaatgcttgaagccgttggaagggtactcagccgtttatatttcgcatatttgcactcgaggagcattcccgatgttagcgcatgaccctcgaaatattaacatactttgctttgaacatcataatcagtgggagcacgctaacacccgaaagggaatgcgtatttatcaagaaaatttagagaaaataaaagtcctcaaaagggacagtttaaaattgcaaaagaaatgaaattagtaaaatttaaggttaaaaacggacaacacattttggttaatgccaattgtgttacgtctattaccaaaaatacaaacgatactacgaatgttaaatgtgctggttctgattgtccttttatcgttttgggtagtattgatgaagtcgaaaagacattagtcaagggaagtaaagtagattcaatagccggcataatggttattttattcataggtatttatatattatcaagtattctaaatttattttaatatgaacttaaacaaaattcagttgattggtagggtctgcaatgatcctcaagtaaaaacgttcgataatgggggtaagatttgcaacgtgtctatcgctacaaacgaacgtgcatataaaacgagtagcggcgttgaagttccggaacgaacagattttcacaacgtggtattcaaaggaggtttagcaggtatatgtgagcagtatgttgctaaaggtatggagttatacgtagagggtactttgcattatcggaagtacacagactcgaataacgtagaaaagactattgcggaaattatcgtttctaacatgcagatgggagcaaagcctggaggcaatagcggtcagcgggcggaatctgccgggagtgggggtcagccgccagtacaaccgacaccgccaccatcacaagccttaaaagaacagcaatttgcagatgatttgccattctaaaaaaagggggatactctaaagcagggtatccctttttgtgttaaataagtgttaaagattaaatttgtaattagaatattaaagttttgctttatatttgcaatgtcaaaaggaaacaaagtaataacaattaaaaattaaccaatatgacaacttacatttataaaggagaaaagatcagtcattcaaagttattaatgcttttgcgttctgcacacgtttttggcggaaacaagctatcacattacgaggctttagtgaaagcggctgagaatgggaacgagcgtgcaacaaatatccttagagatttagaagtaaagtaataacaattaaatagtagaattatggaaaaaagaagaatgtccggagagtttaagatttcggttaatagactaaacgaaagtactgggttatacgaagacggtgtaagcgttgtaactgggtttatgtaccaaatcggtgcttatcaatattttctacactgggataaaacatttcagagggtagatataacagagtcaagtacagggtttagagtgaaaagtattgagaaattgaatggtgagacacctaaacagtgccatgatagagcaatagaggaaatgaagggttttaacccgtctttgggtaactgggaaaaagctaaatcaatgatgaagaaagccggaataccttacccgttgaacgaatggataactaacttaaaggacataaattcaaatgaagaaatcaagaaagattaatcgacttaaaagtcgttttgttacggctaaatttagcttaaaggaacaaagacagtttaccaatattttgagtgaaaaaagaaaaattagaaaggatagggataaaaccttcgattctcttttagaacttgataaaattatattgaaagctgatttatactgtgcaggtttgggtgaatttgtgttttctgagagtgaaatggaggcgtttagcaggtttgaaaaagtaaaaagggagttttatcatggaaagtaaattaaatcagttagaagtccttattttgcaggaagaagaagcatacaaggacatgcgagtagcagagaaaagacatgccgcatgtttacaagaattgtcggaatttgaaagcaaagctaaattaggaaacattcgatattgcggaaactgtgtgtattttccaagtgcgaaaagaggaggaaagtataagtgttcgttgaccggagagaagaaagattattgcagtgagggatgtgaaaaatatagtgaattaccgttttaaaaatagaaaaatatgaataatttgattaaagttagcgaaactataaacgcaaaagaaactatgtgttcaactgaaattgcggaattaacaggtaggagacatgataacatcataagagacataagggggttattatcacagggcgttgccgtcctcaattttgaggaggggacatataaggatgctaataaccaaaatagaccttgttacaatcttacaaagaagggttgtttaattctcgcttccggatatgatgctttattgcgtgaaaagataattaacagatgggaagaacttgaaaaggagaagcgtttcggcaacttcgttattccatctacatttagcgaggctttaatgttagcagccaaacaagcggaagaaatagaggagaaaaataaattgctgttagagcagactccaaaggttgagttttataatgctgttacaggtagcgaggacactattgatatgcgaacggttgccactgtgttgaatatgggaataggtcgtaataaaatatttgaggtattaagagataaacatgttcttgatcgtaaaaacatgccttatcaaaagtatatagatttgggatattttagaaccgttgaaactcaatatacaaaaagtgatgggaccaactgcattaatataaaaactgtggtgtttcaaaagggcttagattttatatgcaaaacattaactttaaataaataagatatgattgattttagcaaaagcgttatttttttaacaaaagaatgtaaagaacagcatgaaagaatgaaggaaaaaggtttccatgatagaaatgtttctttgccggaaatattcgggcttatcatttcggagatgtgcgaggcgatggaggcggaaagaaagggaagaactgtggaaaacggaaagtacacctgggtgttatcatataaggaagatgaaagtttcaagtcgaaatttaatcaatgcataaaagatactgttagcgatgaacttgcagatgtatttatccggtgtttggacgctaccggtaaatataacgaggataataacgatatagtcctatttaaacactgtattgatgaacgtgtgaaaattctaaaaaaaactccgaatacattcgcttattacgtatataatctcgcatactgggtaacaagtaatgaaaaaatatgctgcaattactttactactataatggagatatgtgcagcaatagcaatcatacataacattgatttaggtagagcgattgaggcaaaaatacggtataatgaaacaagaggctacaagcatgggaagaaatactaataacaaaatagataaaaatatgaatttagaacaaatcaagattaacgctattttagcggctgatccggaaggtattcaaatagacggtaatcactatcaaggtgattctattcctttgtatcagtttctcaatcagaataatgttaatacaatggatggctatgtgataaaatatgcattccggcatagacgtaaaaacaaagaaagagatattgcgaaagctattcatactctacaattgattttgaaagacgaatacaacatgtatatgttaggcggacagttgtacacgaaagagcaatatgatgaattgctcagccaggcgaaaagagaggcggaaggaaaagaaactgaaactacggtgatatataccgataaggggatagatattaactctgattctaataggaatattaataggaatatttacgttcgtaaactaagcgaagttcaagccgtttgtatagatagaactaacatttcacttgacgatttgcgggacatggggcttgtgcttaatgcttacgaaatgggaaagggggcatatgttaccagccaaaacggaaaggagttttatgttaaatatggatcctacatttgcctctcagaggatgggtattatcaagtttatagcaagagtgaatttaaaaaactgtttgagcctaaacaataataaataaaatgtaaattaattcaatacgtgattaaaaatgaatgaatcggtagatatatttggaaataaggtcgctttcataaaaccatcattaagcaaagaaaaagaaaagacgctttttgatgactacaaaaactttgtggataaatttaaaacaaagaaaactacagatgattgctacactccgcctgaggtttataattgcattttgagatacgtttcagaaaaatgtaatataagaggagcggaaatagtacgtccattctatccgggtggagattatgaaaattgtgaatacccggatggatgtatcgttgtcgataatcctcctttctctattatttcacagatagtaaagttttatatagaaaaaaatattaaattctttctgttcgctccacacctgacgctattctcccaaagggtcgaatgttgccgaatagtagttggagcatcaatcacctatgaaaatggagcgaatgtaaaaacatcgttcatatccaatttattcgatgatgttacggtaatgtctgatccgtttctttacagagagctcgaaagaataaacgaatcgaaaaaagtgatgcttccaaggtataagtatccaaacagcgttctaaccgtctcagacatgcaatggtgcgtagaacgtggtgtttcaatgcgatttaagaaagatgatatctaccacattaggggacttgatagccaaaagccacataagaaaggtatattcggtgcaggattccttctttcagagagggcggcggcagagagggcggcggcagagagggcggcggcagagaaggcggcggcagagaaggataatattataatttgggaactgtccgaaagagagaaggaaattataaaatcactgggtgaataaacctcaatatactcaaaaagtttaaacctgaatgaaaccacgttgtaagtgttacagcgtggttttctttttgcattcaatttaatacacctatatttgcatagacaaacaattcatttttagcaaagtgaatctatttttgtttaaatataattcttaatgatatggttaaagtaggtagtatgattaaatcacaaggaaaaataaagagaacggacgaacaatgggaggctgataaagcacttgaatctgaattgtttctaaagggttattcttatcgcagaatcagagataagatcaatgagcgctacaaagaaatgggaatagatatacaaatatcttatcagtctgtgtataacgacattcagaaatgtttggctgagtggaaacgggaacaattcactaacatagaccagtatgttacacaggaaattcaagcactcgacaatgtggcgagggaggcgtgggaggaatgggaacgttctaagcgtcctaaatgcaaaacgaaatacaggtttaaaactgctgtcgaggtgcaaaaggaaactactacgggtgacccttcgttcttgaatgtcattcttaacgtacagcaaaggaaggcacgtttgttgggatatgatagtccgttagttgtttctattgtcggggataaagaaaaagagaaaccaaaatacgacttatccagtgttccggaggatgttctcgaaaaaatggcggacgctttgcagaatggaggtgacgatgaagataaataatataccaccagaggaaattgttaagcatgttgcgagaaacaagttcaagaacttcgctaaatacgtagataatggaattatattaagccagtttcataaaacgtattacgaaattcttgatatgtttgcgcatggaaaaataaagaggttaatagtttcgataggaccgcagcacggcaagagtgaaggtagtagccgcaaactacctgcctttatacttggtttacgtccggacgccaagatagcaataggttcttatgctgcaacgcttgctgagggcttcaataaggacatacagcgcattttagatacaccggagtacattagcttgttcccaggtactcgtattatgggagcagagaaaacatcacgttacgaagcgtacacccgtaatagtaaaatgactgaggttgtaggtcgaaaaggatcggttacggctgtcggtcgttcgggtggtttaactggtaggtccgttaacgttgctatcctcgatgatgtttataaggatcacctcgaagccaactcacctattattagagaagccgcttggaagtggtacactacggttatcagaaagcgtcttgacaataacggtcaggaacttatcgtatttacaagatggcataaagacgatttgataggtcggattgagaagaaagagaaagttattacggtgacgaaatggtctgatcttgataatataccggagggcgcatgggtaaagataaactttcctgcattgaaggttggtgagccgacagaaatagacccacggcatgaaggtgaggcgttgtgggaagagaaacatagcgcaaagaaacttcttgctgagcgtgaacttgacaaggtggaatttgagtgcttgaatcaagggaatccgggaagcgcagagggacagctatacggaaagtttaaaacgtggtctgataagtcagatttcggtgtatttctcggtcgtggtaactataccgattgtgcagatacaggaacggataacctttgcagtatatgctatgataaataccgttcaaaacagccagtttggagcgaaaaggaaaagacatacaagtatttaatattctgccttgtcacggatataatatataccactgacccgatagaggttacacaggtgacagtgccggagatgttaaacagaaacgaaactgagtatgctaacattgaaagtaataacggaggacgttcatttgctgttaatatatcgcctaaaactaaaacctcgatacgatggttctctcagcacaataacaaagaggcgagaattttaacacacgctgcgaatgttactcaatctatcgtcatgccgttcggatgggagtctaaatttccacgtttctacgaggacgttacaggctatctgagggactttaaagcgaacgtacatgatgatgcaccggatacattgactggaattgtagagaaagaagttatgcccgctatcgagcctaaacggagaggtattaagcgtataaactaatagaaagcaaattgtatctatgtttcaaaagaatataggtacatttgcattgttaattaattgtttaactaaaaattaagaaaatatgttgtattgtaattgtccgcttggagaggctttgccggatatcccggttgtaacatgtccggagaacttcggtcagattcagaaagtagtatttcagagattgatgggtaaaacggctgaaaattcaataaccgttgcaactgctaaaacattgggtacttggacggctttactcgctgctaaagacgctactaaaatggtggtttcaccgtacattgctgagccgactgttgaagcgggcgaggctttgacgtatggaggaggaaacgcaacacccggtggcgtagttgagattttggggtctaatagtactccgtttactggtaaattcttgaaaactccacagacagttatcaaggttttgaagcaatttatgtgcgaagttaccggaggactgggagtatatttgattaacggcaacggtcagatcgctgctattaaagaaggtgaaaactataagccaatccccgttgaatcgctgtttgtaggtgatcgcactatcggaggtttggaagcaccggacgcgaatgtaatctcgtggagtttcaaacctaactggtcggataacttggagattttcaaaccggattttaaccctctgacacagttaataccggcttcgccagcagcctaatgaatgctaagaaaacaatggtttccctcacttgtaaagagttgggggaaactcgtttatttgaagttgaacatgctgaacgtcttttgtcaatgtttcccaaaggagggtgggagataacggaaggtgaaagctattatttaaaacaggatgggaaaatcagtcgaagaaatacgggagatattcaaaaaaccgatcaatcggaggtggatacagaaagcgagggagcaggaggaacggatagcctttcacgcaagggtaaaggttgacgatgtacgtacaaagcctgctttagactttcttaaccgggtgaagatgtggatagcaccggacaaatacgagatatttaactctatgttccatttcccggtaaagacaaacgaggttacgaacgagatatttgataaattgagtcgtgtgtttgacggtcggaatcctgcttttaactatcagttcacggactcggaagatcgggatgactgggagtattacagacaagaagtactgaaagaaccgcatgtttgggctactgatggttgggataatttcaaagatagaattaactccgttcttgtaattgatttgccggaggtgcagcagggtgataaaccagagccttatttctatttcgttgatatatcctcagtggtaagttatgaaactactaaggaggataataatcttatgtcgtggataatgttcaagacgaatgatgaaaagttgattcaaatagatgatactttttacagacgttttaaaatcgaaaagaacaattcactcacattggaggttgaaagtacgcacgatttaggttattgcccttcgcgtttcttttggtctgactctatatcattgcaagaaccggatataaagaaaagcccgcttaccaaagtacttgattcgcttgactggtatctgtatcaatcaacggcaaagaaacatcttgatttgtacggtgcttatccgatctattccggatatgaacaagattgcgattatattgcaaacggaggcaaagagaggtgtaacggacacggtttcttaatcggtgataagggtgaatatattgcagacatggacggtcaacctatgaaatgtccagtatgttcctcgaagcggttgagcggtgcaggttcttatgtagagataccagtaccaagcgagcagcaaccggacttgtcagaccctatcaaaatgctaacagctgatgtatctgctttgcagtataacgtgtctgaggaggaacgtttaaagaagaacattattacctctgtgactggcgtagggggtgaggttcagaaagaaacagctgtaaacgagaaacaagtacaggcttcatttgagagtcaaacgacgattctaaaccggatcaaacgaggctttgaggaagcacaatgctttgtagacgcaacagtttgccgactgagatacggaaatactttcgtttcatgttcgataaactacggcactgagttctatatctatacaccggaacaacttgcagagagatacaaagtactcaaagagtcgggagcaagcgaaagcgagttagacgctatgcgtacacagataatcgaaacagagtacaggcatgacccaatacaaatgcaaaggttattaatcttgaaagagattgaaccttattctcacttaacgagggaagaagcaattaatttgtataaagaaaacgttataagtgaggaagatttgcggataaaactaaacttgcctacatttgtgcgtagatttgaaagagagaacatgaatattatagagtttggctctaatattgattactctaataagataaacaaaattttagaaacattaaaacgttatgcaaatgaacagaccattgtacccggagcacccgcttgacaaggtgacagcagaaaactatctgtgtccggataatgaaaaaggtcactatcacgtgattcaagaaaggttgcagtttgaccctgacactggtgcgagagtgttttcgccagtattgcagaaatacagacctttaacttttgaaatgacggtttacccgtatttgagtagaggaggctacaatattagaattgtgcacgatcctagaaagtatgcaaaggatatgcaggaatattcagaacaggttaaaaaagcaaagcaagaacaggctttaaaggaactgagagaacagatcagagaggaagagagaaagaaggttcttgctgagttgaaaaaggaagaaaagaaaggaggtaagtaatgttaacggtagatattctaagacaaaataaagctctttcagagctaacagatgagcagttgaatgctattgctaccctttcgcagaatgacgaaatgcaggttattcagacgaaagttaaggaggaaagagcaaaagcgactttatctctgagtcaagcattcggcattgacgatgttacagaccttacatttgaaaaagctgtcgagtttggaaagaacaaactttcgtctgttgattctgctaagtttgaaaagactattgcagatcttaaaacagaacttgaagccgaaaaagcaaagaaggtaggtgataaggataacgagaaaatagccgctttgcaggctgagttaaacgatactaagacgaagtattcggaactaacaaatcagcttacagagaaagaaaaagagttttcaaacaagctatccgattataagatcacttcccatattacacaggctttaggcagcatgaagtttggaaagggagttaatgacgctatgttgaatattatcaaacagcaagcagtgaacgatcttaaaactcagtttacgcctaccattgtcgagaaagacggtaaggaaagtatcgtgtttatgaaagacggtgtaccttacaacaatccggcaaacggtctaaatccttattctgtatctgagttgctgactgagaaactgaaaccgttcggtgtcctcgatgaaggaagaaccgtgggaggtgcaggaggcaaaggaggcggaaaaggaacgccaaccgcaattgatctgacaggctgtaaaactaaggtagaggcgcaggaggttgcgcataaataccttgccggaaaagggctaacagtggggtcggaagagtaccaaacggaactcaatacaatttggcaagagaatgatattcagaacctgccgttacagtaaaaacaaaggggatacccatatttataaactttaaaaacagattattatgagcttaattgctacacgtacacaggaaatgcggttgagaaatccgcaggtagacaagaacatgagccgcctaacagagtggggtgcgcttgactttttcctttctcagacgaatgcacctgactctatgcttaacgatgaaactaaacgtagggcgtttagttctatggggaccgacatcaaaattccggtgattgattatgacggtaccgtcacagttgcaaacgaacgcacatgtgtcattgcggacgctgagaatacctctaaactgatgacggttgtgtggaaaacatacgctttcggttttacgatggtcccgacgatgtttaacaacaacgaaatcgactatcagaaagattttgaaaagaaaatgcttaaattttctcgtaaattcttggatcaagttgacaaggacgctatcgcagcactggaaacagctaaaacgcagaagttcggcaacctgctttactacactcagtcagcaaacgatgtgcaggtgaattacatgcagcgtaatgacatactcggagatttgcacccgatgttccgttctatggactattccggtcaacttcatatcgtaggtgatacaggtgtagacgctattgttcggaagttggaacaacacggaatctacaacgatgttaacaagcaactggaatatgcaaataagattttccactttacaaataacatggttttggaaagtgaaaactttgctcagttctacgcaattgaaagcggtaatgttggtatgttgacccgtgttgaccgcgaagccctcagacgagcaacctctaaaacaggtcacgaatgggacgttatcaacttcccgtttgcaggtttccaagttggtacacattactacgagtcggtaggtgatcagtcagaaatcgcaggtgcggcaactgctgacatgaaatgtaacatcaaagagcactacggtttctccgttgatattgctttcgttgtagcttacaactctgcaccggcaactgtttccaacccggttatgaaggttgaaattaagaaggacggttctcagtttggcggtacacctgtttacatcacgaatgccgaacagatcggaggcgggtctgctgccagtgaaatgtctgttaatattgctaaaattggaggaaatccagttgcagaatcagctttaaaggtagatttggacaaggttaagggtgcgtcagtttcatctactggtggtgtagttgatgtaaaggttaactctcaggcttcaaatcttaatgtagaggttaaaaacgcagatagcgcaccagttccaacgaaagcagttgggggatagtagttatatagtaaattaaagtttaattaaaagggaggggaaagaaatatcccttcccttttttaatttgaagcaacatgtatagaataaaagacataaaagatagcttaaaaaacgtagtaggttggaggcagtcatacgatttaggcagtcagatagacaccgaactaaccacatctgaaagtggtatttcttatcaagacgttcacccacttgtgacgcttgaaaacatatcatctataatgccgttagactactataagaagtacccggaatacagcgatataaagacttatgtggtcggtgacaaggtgaggtttaatagtgaccctctattgtctaaaccatctgtatggatagccacaaacgaaacaaccggagagcagccctcagagggtagtcaaaattggaagagatataaccctttatctgattacctcagagagttaaacgaaaaagcaataacggctactatcactaaatttatcacgggaaagacgatagcgggggaaacaaagatgctgttagagcgtaggccgctgtttgatggttcgggatactatacaaatcagattgaccctacaaagagcatggtaggctatgaaattttgccagtccgtgcgatgggtgtgactacaaagatagaaaggataggtttgcagtttacgaagccagtgaaagttaagatgtatctttttcacagctcacagcctcagcccatacacacgtttgacttgaattatactgcaaggggtgcgtatcaatggtttgatgtacctgacacatttttaccttatatttctgaggcaacgtcaccgggtggcacatggtatctgtgctacgatcaagaacaattgccgtatgatgtatttgcaatcaacatggcaaaagactttagcgcggagccgtgcggaacttgcaacgttggcagcgtacaggcgtggagagagctaacaaagtacattcaaatatcaccgttcaggaatgactcaaaacagggtgagaatctgtttaacattcaatcaagcgtctatacacctgcaacatgctacggtattaacgttcagtttacggtagcttgtgacatcaccgacttcattgtaagcgaaagacttgtgtttgcgaacgctatttctttgcagatggctgcttatatccttcgtgaacttgccttgaatccgaacgtccgacagaatgctaatcagttgaacattgatagagaatcaatcttatacgaggttgacggtgtgtcgcaaggacgtgcgcagggtatcgggcaccagttaaatcaagcaatgaaagcgttaagcgtagatactaaaggtatggatagaatatgcttgacttgccggaatggtggtatcagatttaaatcgacatgataacaaacttattagatagagttaagaaagtgaaagaggctttagactcaggacgaatagcaaaggaaattgtgaggaacaacgataacattcttatcgacatgaacgcacaagaccagctattcgccaaaggggtaaacaggttgggggtcagaatagacgagtatagaccttatagccccttcactatcaaggttaagatagagaagcggcaaccgtatgaccgggtgactttaaaggacacaggggagttttacgactctttttacgttgaaacggctgaggatagattttacattaaagcctcagacgaaaaaacggactggttgattaaaaaatacggtgctgagatatttggtttaactaatgaatcgcttgccgaatttatcagcgattatgtgaaagatgaagcagcaaaaaaagtaaaggagataataaatgaaagatagagctattttaaagccgaatgcggtactttttgatgaagtgataggaaatgtacaggttagccttgtaaagtcgctacaatggctaaattatgcgtttggaagcgcatataagttagtggagcgaacggagaaaggaaagtttgtcactccttctgtttattacaaggataaagactatttgaggttagaaccaaatgacaagtacggcaacacctgttttttctatatccacgactcgcaagattacgaggataacggagcgttcggcttcggtgatctgaaaggtga